(19) United States
(12) Patent Application Publication
(10) Pub. No.: US 2017/0249440 A1
(43) Pub. Date: Aug. 31, 2017

(54) 3D PRINTING SURGICAL REPAIR SYSTEMS

(71) Applicant: ConforMIS, Inc., Bedford, MA (US)

(72) Inventors: Philipp Lang, Lexington, MA (US); Daniel Steines, Lexington, MA (US)

(73) Assignee: ConforMIS, Inc., Bedford, MA (US)

(21) Appl. No.: 15/457,324

(22) Filed: Mar. 13, 2017

Related U.S. Application Data
(63) Continuation of application No. PCT/US2015/050301, filed on Sep. 15, 2015.
(60) Provisional application No. 62/050,280, filed on Sep. 15, 2014.

(51) Int. Cl. G06F 19/00; A61F 2/46; B33Y 10/00; B29C 67/00; B33Y 80/00; B22F 3/105; B23K 26/342; B23K 26/70; A61F 2/30; B33Y 50/02

(52) U.S. Cl. CPC G06F 19/3437; G06F 19/322; A61F 2/30942; A61F 2/4684; B33Y 10/00; B33Y 50/02; B33Y 80/00; B22F 3/1055; B23K 26/342; B23K 26/70; B29C 67/0088; B22F 2003/1057; B29L 2031/7532

(57) ABSTRACT

Various embodiments of methods of making one or more components of surgical repair systems, including embodiments employing 3D printing of one or more components, are disclosed. In various embodiments, multiple surgical repair system components may be based, at least in part, on a patient-adapted surface model.

### REFERENCE TO RELATED APPLICATIONS

This application is a continuation of International Patent Application No. PCT/US2015/050301, filed on Sep. 15, 2015, which claims the benefit of the filing date of U.S. Provisional Application No. 62/050,280, entitled “3D Printing Surgical Repair Systems” and filed on Sep. 15, 2014, the entire contents of each of which are incorporated herein by reference.

### FIELD OF THE INVENTION

The present disclosure generally relates to surgical repair systems (e.g., surgical plan/resection cut strategy, surgical instruments, implants, trial implants) as described in, for example, U.S. patent application Ser. No. 13/397,457, entitled “Patient-Adapted and Improved Orthopedic Implants, Designs And Related Tools,” filed Feb. 15, 2012, and published as U.S. Patent Publication No. 2012-0209394, which is incorporated herein by reference in its entirety. International Patent Application No. PCT/US13/36505, entitled “Devices and Methods for Additive Manufacturing of Implant Components,” filed Apr. 13, 2013, is also incorporated herein by reference in its entirety. In particular, various embodiments of methods of making one or more components of surgical repair systems (standard and patient-adapted) utilizing 3D printing techniques are described herein.

### BRIEF DESCRIPTION OF THE DRAWINGS

FIGS. 1-7 are flowcharts depicting exemplary embodiments for making patient-adapted surgical repair system components;

FIG. 8 is a flowchart depicting a process for generating a model of a patient's joint (and/or a resection cut, guide tool, and/or implant component);

FIGS. 9A and 9B are front and side views of a surface outline for a patient's femur and tibia;

FIG. 10 is a flowchart depicting an exemplary process for determining specifications for patient-adapted surgical repair system components; and

FIGS. 11-27 are flowcharts depicting exemplary embodiments for making patient-adapted surgical repair system components.

### DETAILED DESCRIPTION OF THE INVENTION

Reference will now be made in detail to the present embodiments (exemplary embodiments), examples of which are illustrated in the accompanying drawings. Wherever possible, the same reference numbers will be used throughout the drawings to refer to the same or like parts.

In this application, the use of the singular includes the plural unless specifically stated otherwise. Furthermore, the use of the term “including,” as well as other forms, such as “includes” and “included,” is not limiting. Also, terms such as “element” or “component” encompass both elements and components comprising one unit and elements and components that comprise more than one subunit, unless specifically stated otherwise. Also, the use of the term “portion” may include part of a moiety or the entire moiety.

The section headings used herein are for organizational purposes only and are not to be construed as limiting the subject matter described.

### Applicable Manufacturing Techniques

Various embodiments disclosed herein include methods of manufacturing one or more components of surgical repair systems. Such components can include, for example, implant components, trial implant components, surgical instruments, and anatomical models. Furthermore such components can be patient adapted (i.e., patient specific or patient engineered) or standard (i.e., off-the-shelf, not patient-specific). A variety of manufacturing processes or techniques can be used in the production of such components. In certain embodiments, manufacturing surgical repair system components can include making the components from starting materials, for example, metals and/or polymers or other materials in solid (e.g., powders or blocks) or liquid form. In addition or alternatively, in certain embodiments, manufacturing can include altering (e.g., machining) an existing component, for example, a standard blank implant component and/or guide tool or an existing implant component and/or guide tool (e.g., selected from a library). The manufacturing techniques for making or altering a surgical repair system component can include any techniques known in the art today and in the future. Such techniques include, but are not limited to additive methods (e.g., methods that add material, for example to a standard blank), as well as subtractive methods (e.g., methods that remove material, for example from a standard blank).

Embodiments disclosed herein provide methods utilizing additive manufacturing or 3-dimensional (“3D”) printing, at least in part, for producing one or more components of surgical repair systems. Generally, 3D printing (also sometimes referred to as Solid Freeform Fabrication “SFF” or additive manufacturing) encompasses processes that can be used to create three-dimensional, physical parts, or objects, from digital models using an additive process, i.e., a process in which successive layers of material are laid down and solidified in pre-determined shapes. As such, a typical 3D printer or 3D printing apparatus can be considered a type of computer controlled industrial robot capable of executing the steps of an additive manufacturing process. As described further below, there are currently several different additive manufacturing processes that a 3D printing apparatus may be configured to execute. Various embodiments disclosed herein, however, are not necessarily restricted to particular additive manufacturing processes. Thus, except where explicitly indicated otherwise herein, “3D printing apparatus” is intended to generally encompass apparatuses capable of executing one or more of the currently developed additive manufacturing processes, as well as future apparatuses configured to execute newly developed additive manufacturing processes.

The digital models from which a part may be built by a 3D printing apparatus can include a variety of forms or formats of electronic or computerized data files that describe the part, depending upon, for example, the particular 3D printing apparatus, additive manufacturing process, and/or software to be used. But, generally, the digital models may be, at least initially, provided in the form of one or more computer-aided design (CAD) files (e.g., STL, DWG IGES, VDA). In some embodiments, at least some of the component specifications and/or electronic models (e.g., CAD files) may be transferred into one or more software-directed computer systems that perform a series of operations to combine, transform, supplement, covert, and/or otherwise process the data into manufacturing specifications of one or more particular forms and/or formats. One or more of these operations may be partially or fully automated by software, and/or one or more of these operations may be performed manually by an operator.

In some embodiments, the electronic models and/or manufacturing specifications may be transferred by a user and/or by electronic transfer (automatically or manually) into a software-directed computer system that directs one or more manufacturing instruments and/or industrial robots (e.g., 3D printers) to perform one or more manufacturing steps. In some cases, the one or more software-directed computer systems that directs the manufacturing step(s) may be the same one or more software-directed computer systems that generated and/or processed the component specifications and/or electronic models. Further, any one or more of the various software-directed computer systems may be integral with or may be virtually connected (e.g., in communication) to the manufacturing instruments and/or industrial robots (e.g., 3D printers). Accordingly, as used herein, providing information (e.g., a surface model, a component model, an STL file) to a 3D printing apparatus or other manufacturing apparatus (e.g., computer numerically controlled CNC machine tools) includes providing such information to corresponding integral and/or virtually connected computer systems.

By way of example, for some 3D printing apparatuses, initial CAD file data may need to be combined, processed, transformed, supplemented, and/or converted by one or more software applications into one or more other electronic files, forms, and/or formats that can be used to control the additive manufacturing process. For example, in some embodiments, CAD models/data may need to first be prepared (e.g., imported, repaired, oriented, positioned) in a scene or plan appropriate for the particular 3D printing process and/or apparatus. This may be accomplished using, for example, a Rapid Prototyping (RP) software application, which may be off-the-shelf software such as Magics (Materialise, 44650 Helm Court, Plymouth, Mich. 48170) and/or a custom software application. Further, depending upon the particular 3D printing process and/or apparatus, support structures may need to be added to or associated with the parts or components in the CAD models, as discussed in greater detail below. Additionally, part data (e.g., STL format) may need to be transformed into layer data (e.g., SLI format), which may be done utilizing the same software application or another software application, which may be off-the-shelf software such as EOS RP-Tools (EOS GmbH, Robert-Stirling-Ring 1, 82152 Krailling/Munich, Germany) and/or a custom software application. Furthermore, in some cases, layer data may need to be combined, edited, and/or converted into data/instructions for building in a final job file format that may be executed by the 3D printing apparatus.

In addition to using 3D printing techniques, various other types of manufacturing techniques, including traditional techniques, can be utilized in embodiments disclosed herein for producing components of surgical repair systems. Various disclosed embodiments can include utilizing a single manufacturing technique or combinations of one or more of the manufacturing techniques disclosed herein (e.g., a first step in producing a component can utilize a first technique, a second step can utilize a second technique, and, optionally, so on). Some embodiments can include utilizing one or more 3D printing techniques for producing all of the components of a system. Alternatively, in some embodiments, a 3D printing technique may be utilized, alone or in combination with other techniques, for producing one or more components, while only non-3D printing techniques may be utilized for producing other components of the same system. Exemplary manufacturing techniques, including both 3D printing and other classes of techniques, that can be used in various embodiments for producing components of a surgical repair system (e.g., implant components, trial implant components, guide tools) are summarized in Table 1 below.

TABLE 1
Exemplary techniques for producing surgical repair system components.

| Technique | Brief description of technique and related notes |
|---|---|
| CNC | CNC refers to computer numerically controlled machine tools, |
| | a computer-driven technique, in which machine tools are driven |
| | by one or more computers, e.g., by computer-code instructions. |
| | Embodiments of this method can interface with CAD software |
| | to streamline the automated design and manufacturing process. |
| CAM | CAM refers to computer-aided manufacturing and can be used |
| | to describe the use of software programming tools to efficiently |
| | manage manufacturing and production of products and |
| | prototypes. CAM can be used with CAD to generate CNC |
| | code for manufacturing three-dimensional objects. |
| Casting, including | Casting is a manufacturing technique that employs a mold. |
| casting using rapid | Typically, a mold includes the negative of the desired shape of |
| prototyped casting | a product. A liquid material is poured into the mold and |
| patterns | allowed to cure, for example, with time, cooling, and/or with |
| | the addition of a solidifying agent. The resulting solid material |
| | or casting can be worked subsequently, for example, by sanding |
| | or bonding to another casting to generate a final product. |
| Welding | Welding is a manufacturing technique in which two |
| | components are fused together at one or more locations. In |
| | certain embodiments, the component-joining surfaces include |
| | metal or thermoplastic and heat is administered as part of the |
| | fusion technique. |
| Forging | Forging is a manufacturing technique in which a product or |
| | component, typically a metal, is shaped, typically by heating |
| | and applying force. |
| Rapid prototyping | Rapid prototyping refers generally to automated construction of |
| | a prototype, typically using an additive manufacturing |
| | technology, such as EBM, SLS, SLM, SLA, DMLS, 3DP, FDM |
| | and other technologies, but can also be used to refer to using |
| | such techniques for producing a final product. |
| EBM | EBM refers to electron beam melting, which is a powder-based |
| | additive manufacturing technology. Typically, successive |
| | layers of metal powder are deposited and melted with an |
| | electron beam in a vacuum. |
| SLS | SLS refers to selective laser sintering, which is a powder-based |
| | additive manufacturing technology. Typically, successive |
| | layers of a powder (e.g., polymer, metal, sand, or other |
| | material) are deposited and melted with a scanning laser, for |
| | example, a carbon dioxide laser. |
| SLM | SLM refers to selective laser melting, which is a technology |
| | similar to SLS; however, with SLM the powder material is fully |
| | melted to form a fully-dense product. SLM is also variously |
| | referred to by the trade names DMLS or LaserCusing. |
| SLA or SL | SLA or SL refers to stereolithography, which is a liquid-based |
| | additive manufacturing technology. Typically, successive |
| | layers of a liquid resin are exposed to a curing, for example, |
| | with UV laser light, to solidify each layer and bond it to the |
| | layer below. |
| DMLS | DMLS refers to direct metal laser sintering, which is a powder- |
| | based additive manufacturing technology. Typically, metal |
| | powder is deposited and melted locally using a fiber optic laser. |
| | Complex and highly accurate geometries can be produced with |
| | this technology. This technology supports net-shaping, which |
| | means that the product generated from the technology can |
| | require little or no subsequent surface finishing. |
| LC | LC refers to LaserCUSING ®, which is a powder-based additive |
| | manufacturing technology. |
| 3DP | 3DP refers to three-dimensional printing, which is a high-speed |
| | additive manufacturing technology that can deposit various |
| | types of materials in powder, liquid, or granular form in a |
| | printer-like fashion. Deposited layers can be cured layer by |
| | layer or, alternatively, for granular deposition, an intervening |
| | adhesive step can be used to secure layered granules together in |
| | bed of granules and the multiple layers subsequently can be |
| | cured together, for example, with laser or light curing. |
| LENS | LENS refers to Laser Engineered Net Shaping, which is a |
| | powder-based additive manufacturing technology. Typically, a |
| | metal powder is supplied to the focus of the laser beam at a |
| | deposition head. The laser beam melts the powder as it is |
| | applied, in raster fashion. The process continues layer by layer |
| | and requires no subsequent curing. |
| FDM | FDM refers to fused deposition modeling is an extrusion-based |
| | additive manufacturing technology. Typically, beads of heated |
| | extruded polymers are deposited row by row and layer by layer. |
| | The beads harden as the extruded polymer cools. |

In some exemplary embodiments, the 3D printing techniques of Selective Laser Sintering (SLS) and Direct Metal Laser Sintering (DMLS) (also sometimes referred to as Selective Laser Melting (SLM)) can be employed to produce the components of a surgical repair system. For example, in some embodiments, a model for a patient-adapted implant component can be provided to a DMLS printing apparatus to print a metal implant component corresponding to the model. Additionally, a model for a patient-adapted instrument and, optionally, a patient-adapted trial implant and/or a patient-adapted anatomical model may be provided to one or more SLS printing apparatuses to print the instrument and optional trial implant and/or anatomical model out of a polymer (e.g., nylon).

In various exemplary embodiments, the DMLS apparatus can utilize a raw material comprising a CrCo powder having an average particle size of between 34 and 54 microns, although larger and/or smaller particles may be used with varying degrees of utility (as well as the use of differing size particles in creating a single implant component). In various embodiments, the deposed particle layer may be approximately 60 microns thick which, when melted, consolidated and cooled, can create a solid structural layer of approximately 20 microns thickness.

Alternatively or in addition to the nylon and CrCo exemplary materials noted above, any material known in the art can be used for manufacturing the surgical repair components described herein, for example, including, but not limited to, metals (including metal alloys), ceramics, plastic, polyethylene, cross-linked polyethylene, polymers or plastics, pyrolytic carbon, nanotubes and carbons, bioplastics and/or biologic materials.

For example, a wide-variety of metals can be useful in the practice of the embodiments described herein, and can be selected based on any criteria. For example, material selection can be based on resiliency to impart a desired degree of rigidity. Non-limiting examples of suitable metals include silver, gold, platinum, palladium, iridium, copper, tin, lead, antimony, bismuth, zinc, titanium, cobalt, stainless steel, nickel, iron alloys, cobalt alloys, such as Elgiloy®, a cobalt-chromium-nickel alloy, and MP35N, a nickel-cobalt-chromiummolybdenum alloy, and Nitinol TTM, a nickel-titanium alloy, aluminum, manganese, iron, tantalum, crystal free metals, such as Liquidmetal® alloys (available from LiquidMetal Technologies, www.liquidmetal.com), other metals that can slowly form polyvalent metal ions, for example to inhibit calcification of implanted substrates in contact with a patient's bodily fluids or tissues, and combinations thereof.

Suitable synthetic polymers include, without limitation, polyamides (e.g., nylon), polyesters, polystyrenes, polyacrylates, vinyl polymers (e.g., polyethylene, polytetrafluoroethylene, polypropylene and polyvinyl chloride), polycarbonates, polyurethanes, poly dimethyl siloxanes, cellulose acetates, polymethyl methacrylates, polyether ether ketones, ethylene vinyl acetates, polysulfones, nitrocelluloses, similar copolymers and mixtures thereof. Bioresorbable synthetic polymers can also be used such as dextran, hydroxyethyl starch, derivatives of gelatin, polyvinylpyrrolidone, polyvinyl alcohol, poly(N-(2-hydroxypropyl) methacrylamide) (HPMA), poly(hydroxy acids) (PHA), polycaprolactone (PCL), poly(epsilon-caprolactone), polylactic acid (PLA), polyglycolic acid (PGA), poly(dimethyl glycolic acid), poly(hydroxy butyrate) (PHB), and combinations thereof.

Other appropriate materials include, for example, the polyketone known as polyetheretherketone (PEEK). This includes the material PEEK 450G, which is an unfilled PEEK approved for medical implantation available from Victrex of Lancashire, Great Britain. (Victrex is located at www.matweb.com or see Boedeker www.boedeker.com). Other sources of this material include Gharda located in Panoli, India (www.ghardapolymers.com).

It should be noted that the material selected can also be filled. For example, other grades of PEEK are also available and contemplated, such as 30% glass-filled or 30% carbon filled, provided such materials are cleared for use in implantable devices by the FDA, or other regulatory body. Glass filled PEEK reduces the expansion rate and increases the flexural modulus of PEEK relative to that portion which is unfilled. The resulting product is known to be ideal for improved strength, stiffness, or stability. Carbon filled PEEK is known to enhance the compressive strength and stiffness of PEEK and lower its expansion rate. Carbon filled PEEK offers wear resistance and load carrying capability.

As will be appreciated, other suitable similarly biocompatible thermoplastic or thermoplastic polycondensate materials that resist fatigue, have good memory, are flexible, are deflectable, have very low moisture absorption, and/or have good wear and/or abrasion resistance, can be used. The implant can also be comprised of polyetherketoneketone (PEKK).

Other materials that can be used include polyetherketone (PEK), polyetherketoneetherketoneketone (PEKEKK), and polyetheretherketoneketone (PEEKK), and, generally, a polyaryletheretherketone. Further, other polyketones can be used as well as other thermoplastics.

Components to be formed from polymers can be prepared by any of a variety of approaches including conventional polymer processing methods. Preferred approaches include, for example, injection molding, which is suitable for the production of polymer components with significant structural features, and rapid prototyping approaches, such as reaction injection molding and stereo-lithography. The substrate can be textured or made porous by either physical abrasion or chemical alteration to facilitate incorporation of the metal coating. Other processes are also appropriate, such as extrusion, injection, or compression molding and/or machining techniques. The polymer may be chosen for its physical and mechanical properties and its suitability for carrying and spreading the physical load between the joint surfaces.

More than one metal and/or polymer can be used in combination with each other. For example, one or more metal-containing substrates can be coated with polymers in one or more regions or, alternatively, one or more polymer-containing substrate can be coated in one or more regions with one or more metals.

Finally, in addition to manufacturing physical components, optionally, in some embodiments, implant, implant trial, and/or guide tool design specifications and/or electronic models, as well as surgical plan specifications and/or associated electronic models may be transferred by a user and/or by electronic transfer into a software-directed computer system that performs a series of operations to transform the data into one or more surgical procedure specifications or instructions. In some instances the same or another software computer system may be configured to use the surgical procedure specifications or instructions to direct one or more automated surgical instruments, for example, a robot, to perform one or more surgical steps. In certain embodiments, one or more of these actions can be performed as steps in a single process by one or more software-directed computer systems.

### 3D Printing Surgical Repair System Components

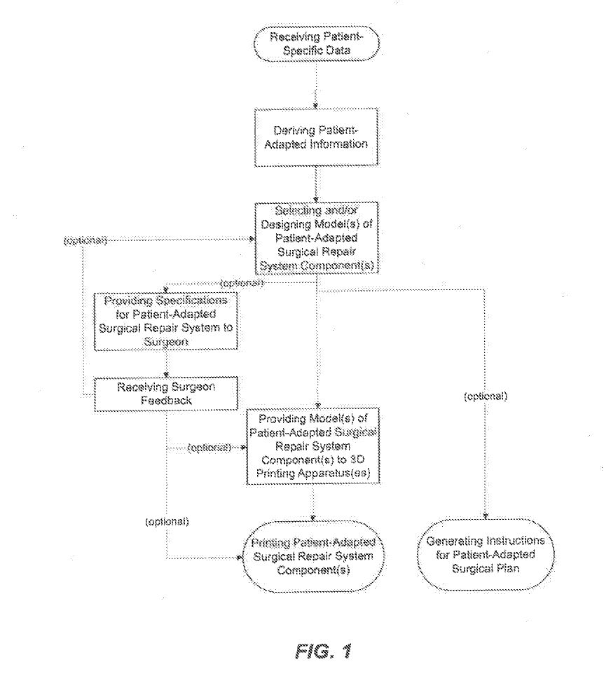

Various embodiments disclosed herein provide pioneering methods for just-in-time manufacturing and/or mass-customization (large scale production of individualized patient-adapted components) of surgical repair systems using 3D printing. FIG. 1 provides a flowchart of general steps utilized in some embodiments of such advanced methods for manufacturing surgical repair systems, and further detailed descriptions of aspects of these steps are provided herein below. As shown, the manufacturing process can begin with receiving patient-specific data (e.g., one or more of the examples of patient-specific data described in detail below), which can be associated with a joint or other biological structure to be treated. Then, patient-adapted information can be derived from the patient-specific data. The derived patient-adapted information can be used for selecting and/or designing one or more aspects of components of a patient-adapted surgical repair system. Components of the surgical repair system can include, for example, implant components, trial implant components, surgical instruments, physical anatomical models, and/or a surgical plan for treating the patient's joint or other biological structure. Models (e.g., electronic) for the one or more selected and/or designed components may then be generated and/or provided to one or more 3D printing apparatuses and the respective components may be printed. In some embodiments, derived patient-adapted information may also be utilized in other steps of manufacturing components of the patient-adapted surgical repair system (e.g., generation of support structures, removal of support structures, surface-finishing processes, inspection processes), as discussed further below. A variety of different 3D printing processes can be used for printing implant components, trial implant components, and/or instruments, and examples of such processes are described further below. Optionally, patient-adapted surgical plan instructions may also be generated.

As additionally shown in FIG. 1, some embodiments may also, optionally, include providing specifications for at least a portion of the selected and/or designed surgical repair system to a surgeon for review, prior to producing at least some of the final components. The specifications provided to the surgeon may include one or more electronic and/or physical models of at least a portion of the patient-specific data, of the derived patient-adapted information, and/or of one or more of the components of the selected and/or designed surgical repair system. After considering the specifications, the surgeon may then provide feedback regarding it. If the planned surgical repair system is approved by the surgeon, some or all of the proposed final components of the surgical repair system may be produced (e.g., 3D printed). If the surgeon's feedback indicates one or more changes are needed, further selecting and/or designing of one or more components of the surgical repair system may be performed, optionally, incorporating one or more changes indicated by the surgeon. Then, optionally, the revised specifications for the surgical repair system may again be provided to the surgeon for review and feedback (optionally, this process may be repeated until a revised set of specifications for the surgical repair system are approved by the surgeon), or models for the revised one or more selected and/or designed components may then be generated and/or provided for production (e.g., provided to one or more 3D printing apparatuses). Various embodiments disclosed herein advantageously enable both obtaining such surgeon review and/or suggested modifications of specifications for a patient-adapted surgical repair system and producing the corresponding final components in a timely and efficient manner. In particular, various embodiments' provision of methods for 3D printing of at least some (and in some embodiments all) of the patient-adapted components for a surgical repair system and utilization of forms of the same derived patient-adapted surface model for multiple steps in the production of such components can enable manufacturing patient-adapted surgical repair systems more quickly and/or more cost efficiently than previously attainable.

As noted, some of the more specific methods developed for use in additive manufacturing of surgical repair systems can include deriving at least one patient-adapted surface model that can be used in producing multiple components of the surgical repair system. In some cases, this technique of using one or more common, patient-adapted surface models can have a variety of advantages, including, for example, streamlining selection, design, and/or modeling of components, by reducing the number of patient-adapted surface models needed to be produced and/or the work required to produce various forms of such models that are required for one or more steps of manufacturing a patient-adapted surgical repair systems. Thus, manufacturing time and costs may be reduced. Such techniques may also result in systems with components that function better together and/or better reproduce an intended treatment result.

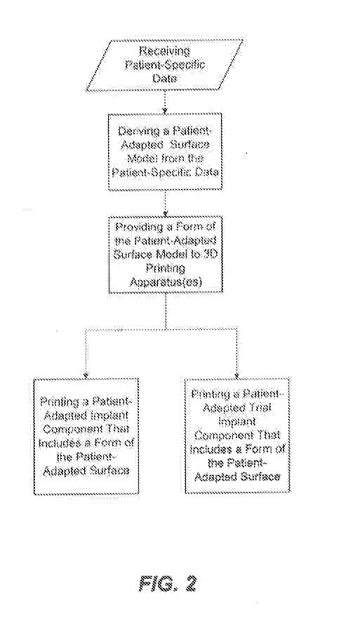

For example, as shown in FIG. 2, in some embodiments, a patient-adapted implant component and a patient-adapted trial implant component may each be produced utilizing, at least in part, one or more forms of the same derived patient-adapted surface model. In particular, some such embodiments can include first receiving patient-specific data associated with a joint or biological structure of a patient to be treated. At least one patient-adapted surface model may then be derived (e.g., utilizing one or more of the techniques described below for deriving surface models) from, at least in part, a portion of the patient-specific data. In some embodiments, an electronic model of an implant component and an electronic model of a trial implant component may each be generated and/or modified such that each includes a form of the derived patient-adapted surface model. By way of example, in some embodiments the patient-adapted surface model may be utilized as corresponding surfaces (e.g., a joint facing surface, a bone facing surface, a bone-cut surface) in both the model of the implant component and the model of the trial implant component. To produce the final components, the derived patient-adapted surface model (optionally, along with other information and/or surface models) may then be provided to one or more 3D printing apparatuses. For example, in some embodiments, the derived patient-adapted surface model may be provided to the 3D printing apparatus(es) as part of electronic models for each of the implant component and the trial implant component. Using, at least in part, the provided models, a patient-adapted implant component that substantially includes a form of the patient-adapted surface and a patient-adapted trial implant component that substantially includes a form of the patient-adapted surface may each be printed. As an example, in some embodiments, the model for the patient-adapted implant component can be provided to a DMLS printing apparatus to print the implant component in metal (e.g., CoCr), and the model for the patient-adapted trial implant may be provided to one or more SLS printing apparatuses to print the trial implant out of a polymer (e.g., nylon).

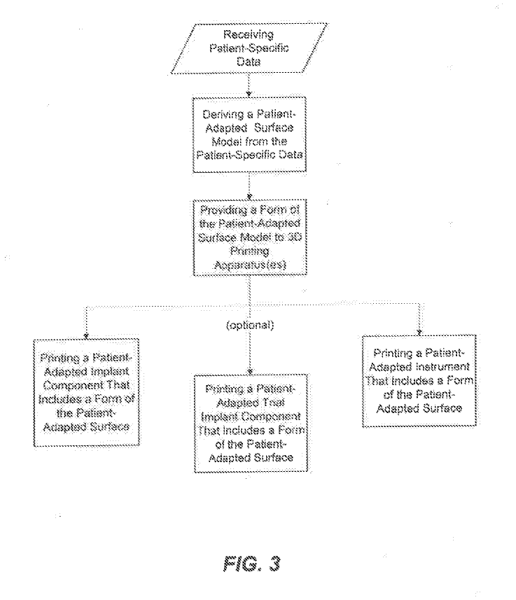

Furthermore, in some embodiments, a patient-adapted implant component, a patient-adapted instrument, and, optionally, a patient-adapted trial implant component may each be produced utilizing, at least in part, one or more forms of the same derived patient-adapted surface model. For example, as shown in FIG. 3, such embodiments can include receiving patient-specific data associated with a joint or biological structure of a patient to be treated. At least one patient-adapted surface model may be derived from, at least in part, a portion of the patient-specific data. A form of the derived patient-adapted surface model may then be provided to one or more 3D printing apparatuses for printing the patient-adapted implant component, the patient-adapted instrument, and, optionally, the patient-adapted trial implant component. In some embodiments, substantially the same form of the derived patient-adapted surface model may be provided for printing each of the components. While in other embodiments, the form of the patient-adapted surface model provided for printing at least one of the components may be substantially different (e.g., positive v. negative and/or corrected v. uncorrected, as described further below) in one or more ways than a form of the patient-adapted surface model provided for printing at least one of the other components. Ultimately, a patient-adapted implant component that substantially includes a form of the patient-adapted surface; a patient-adapted instrument that substantially includes a form of the patient-adapted surface; and optionally, a patient-adapted trial implant that substantially includes a form of the patient-adapted surface may each be printed for the surgical treatment.

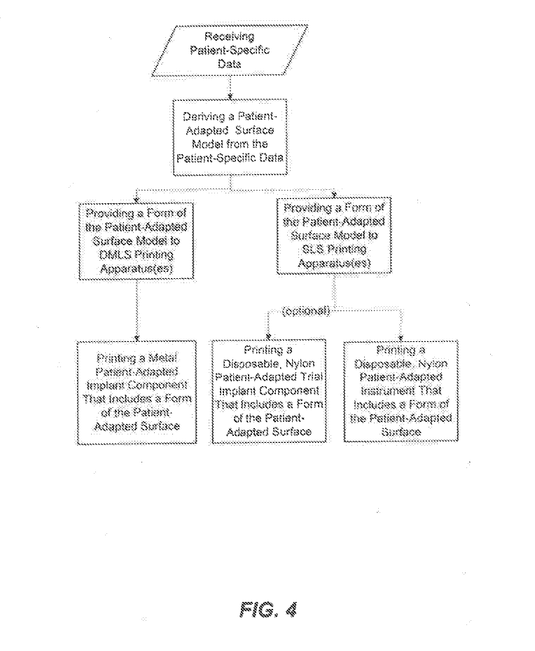

For example, as illustrated in FIG. 4, in some embodiments, a form of a patient-adapted surface model can be provided to a DMLS printing apparatus to print the implant component in metal (e.g., CoCr). Additionally, a form of the patient-adapted surface model can be provided to one or more SLS printing apparatuses to print the instrument and optional trial implant out of a polymer (e.g., nylon).

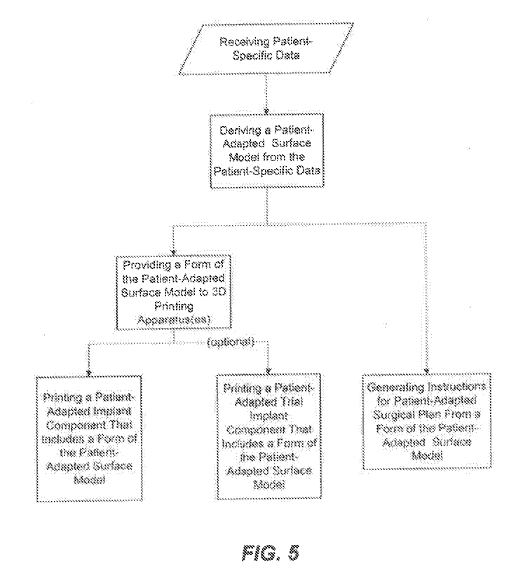

In some embodiments, for example as shown in the flowchart of FIG. 5, a patient-adapted implant component, instructions for a patient-adapted surgical plan, and, optionally, a patient-adapted trial implant component may each be produced utilizing, at least in part, a form of the same derived patient-adapted surface model. Specifically, some such embodiments can include receiving patient-specific data associated with a joint or biological structure of a patient to be treated. Then, at least one patient-adapted surface model (e.g., of a joint facing surface, a bone facing surface, or a bone-cut surface) may be derived from, at least in part, a portion of the patient-specific data. Instructions for a patient-adapted surgical plan may then be generated based, at least in part, on a form of the derived patient-adapted surface model. Additionally, electronic models of a patient-adapted implant and, optionally, a patient adapted trial implant that each includes a form of the derived patient-adapted surface model may be provided to one or more 3D printing apparatuses. Accordingly, a patient-adapted implant that substantially includes a form of the patient-adapted surface may then be printed, and implantation of the patient-adapted implant can be performed according to, in conjunction with, and/or subsequent to execution of the instructions for the patient-adapted surgical plan (e.g., by a surgeon, by a surgical robot). Optionally, a trial patient-adapted implant may also be printed to, optionally, be used during the surgical procedure.

As noted above, in some embodiments, the form of a surface model provided for producing at least one component of a surgical repair system may be different in one or more ways than a form of the same surface model provided for producing at least one of the other components of the system. As one example, the same surface model can be said to have a positive form and a negative form. In some embodiments, the difference between a positive surface model and a negative surface model can be determined by which of two substantially opposing faces of the same surface is identified as the exterior face of the surface and which is identified as the interior face of the surface. In certain instances, this may be information included in and/or accompanying the surface model, while in other instances this may simply be determinable (automated and/or manually) based on the context from which the surface model was derived and/or within which the surface model is used. As a simple illustrative example, a positive form of a surface model of a hemisphere may be described, in part, as a generally convex surface having a particular single radius of curvature. And a corresponding negative form of the same surface model of the hemisphere may be described, in part, as a generally concave surface having the same radius of curvature. Accordingly, a structure having a surface incorporating the negative form of the surface model would be shaped to substantially nestingly receive a structure having a surface incorporating the positive form of the surface model (e.g., as a socket can nestingly receive at least a portion of a corresponding ball). Depending on the shape and/or geometry of a surface model, in some instances (e.g., a surface model that is irregular and/or asymmetric in one or more dimensions) a negative form of a surface may only be able to substantially nestingly receive a positive form of the surface model in a specific position and orientation.

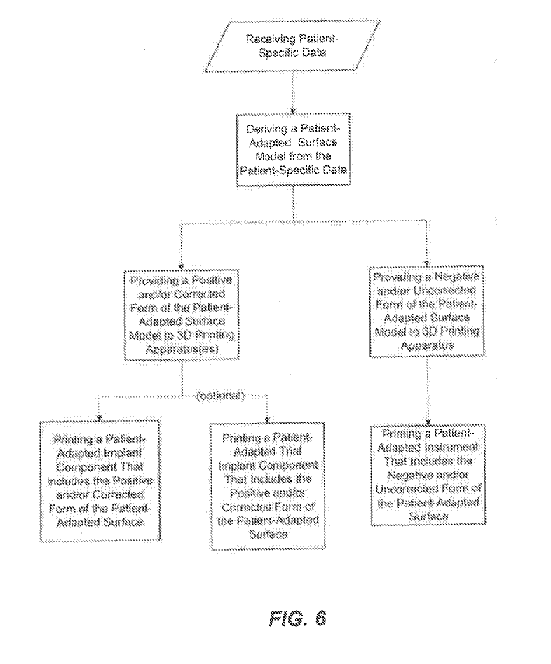

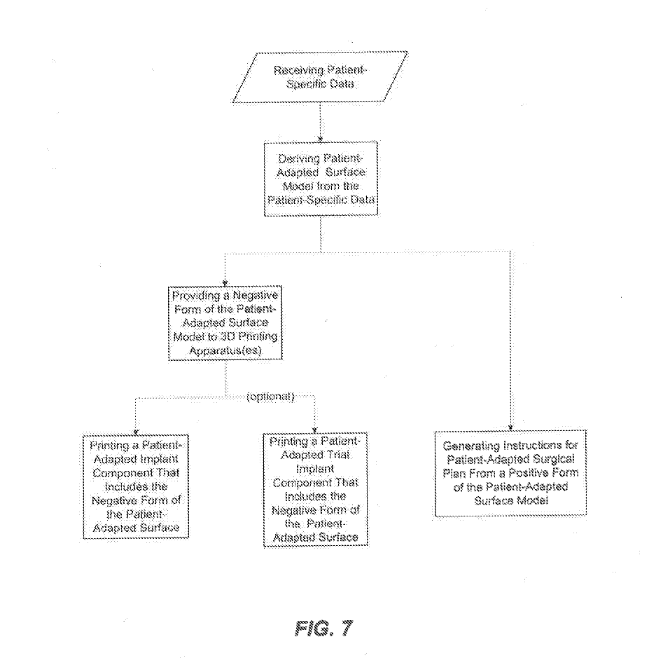

Various embodiments disclosed herein can include utilizing both positive and negative forms of the same patient-adapted surface model (and/or the same standard, i.e., non-patient-adapted, surface model) in producing an articular repair system. For example, as indicated in the flowchart of FIG. 6, in certain embodiments, a positive form of a derived patient-adapted surface model may be provided to one or more 3D printing apparatuses for printing an implant component and, optionally, a trial implant component, while a negative form of the same patient-adapted surface model may be provided to a 3D printing apparatus for printing an instrument. In some such embodiments, the patient-adapted surface model may correspond to at least a portion of an exterior surface of a biological structure (e.g., joint-facing surface, articular surface). Alternatively or in addition, in some embodiments, a negative form of a derived patient-adapted surface model may be provided to one or more 3D printing apparatuses for printing an implant component and, optionally, a trial implant component, while a positive form of the same patient-adapted surface model may be utilized, at least in part, in generating patient-adapted surgical plan instructions, as depicted in FIG. 7. In some such embodiments, the patient-adapted surface model may correspond to one or more planned surfaces or surface portions of a biological structure prepared (e.g., cut-bone surfaces, pin or peg holes) to receive and/or support the implant component.

As will be apparent to those of skill in the art, depending upon, for example, the form and/or source of a derived patient-adapted surface model, the particular surgical repair system components to be produced, and/or the goals for use of the patient-adapted surface, any combination of the use of a positive form of a patient-adapted surface model and a negative form of the same patient-adapted surface model for producing portions of respective surgical repair system components can be employed (e.g., including reversing the use of positive forms and negative forms in exemplary embodiments described herein). For example, a positive form of a derived patient-adapted surface model may be provided to a 3D printing apparatus for printing a patient-adapted instrument, and a negative form of the same patient-adapted surface model may be derived and provided to one or more 3D printing apparatuses for printing a patient adapted implant and, optionally, a patient adapted trial implant. Furthermore, selecting, deriving, and/or generating a positive and/or negative form of the same surface model may be automated, semi-automated, or manually performed by an operator in a software application.

As a second example of how, in various embodiments, the form of a surface model provided for producing at least one component of a surgical repair system may be different in one or more ways than a form of the same surface model provided for producing at least one of the other components of the system, the same surface model can have one or more corrected forms and an uncorrected form. In some embodiments a corrected form of a patient-adapted surface model may be derived (e.g., utilizing one or more of the methods for optimizing and/or correcting surfaces, features, and/or components described below) from the uncorrected form of the surface model, and the corrected form of the surface model and the uncorrected form of the surface model may both be used in producing aspects of the surgical repair system. For example, in some embodiments, as indicated in FIG. 6, a corrected form of a patient-adapted surface model may be derived and provided to one or more 3D printing apparatuses for printing a patient-adapted implant and, optionally, a patient-adapted trial implant. And an uncorrected form of the derived patient-adapted surface model may be provided to a 3D printing apparatus for printing a patient-adapted instrument. In some such embodiments, the patient-adapted surface model may correspond to at least a portion of an exterior surface of a biological structure (e.g., joint-facing surface, articular surface). Similar to the use of positive and negative forms of a surface model, it will be apparent to those of skill in the art that, depending upon, for example, the form and/or source of the originally derived patient-adapted surface model, the particular surgical repair system components to be produced, and/or the goals for use of the patient-adapted surface, any combination of the use of a corrected form of a patient-adapted surface model and an uncorrected form of the same patient-adapted surface model for producing portions of respective surgical repair system components can be employed (e.g., including reversing the use of corrected forms and uncorrected forms in exemplary embodiments described herein).

As noted above, some embodiments can include deriving a patient-adapted surface model of at least a portion of a joint-facing and/or articular surface of a joint of a patient. Additionally or alternatively, some embodiments can include deriving a patient-adapted surface model of at least a portion of one or more planned, resected-bone surfaces that are intended to be formed during a surgical procedure and to support and receive one or more implant components. As discussed in greater detail below, models of resected-bone surfaces may be derived at a variety of stages in the process of developing a surgical repair system, such as, for example: prior to selecting and/or designing one or more implant components and instruments, in conjunction (e.g., through an iterative process) with selecting and/or designing one or more implant components and instruments, and/or subsequent to selecting and/or designing one or more implant components and instruments.

In various embodiments, including at least some that involve generating instructions for a patient-adapted surgical plan, a model of a biological structure of the patient with modifications (e.g., planned, resected-bone surfaces, pin or peg holes) may be derived/generated, for example, as part of a process of selecting and/or designing (e.g., as described herein below) aspects of a patient-adapted surgical repair system based, at least in part, on patient-specific data. Deriving and/or generating such a model may include deriving a patient-adapted surface model of at least a portion of the biological structure (which can include one or more cut bone surfaces and/or pin or peg holes), and/or a patient-adapted surface model may be derived from such a model. Accordingly, in some embodiments, such a derived surface model may be utilized to generate instructions for preparing the modified biological structure (e.g., instructions for making bone cuts and/or placing pin or peg holes) during the surgical procedure. Furthermore, in some embodiments, a form (e.g., a negative—as indicated, for example, in the embodiment of FIG. 7) of the same derived surface model may be included in an electronic model of an implant component and, optionally, an electronic model of a trial implant component, as the bone-facing surface(s) of the component. In some embodiments, the electronic models of the implant component and optional trial implant component may be provided to 3D printing apparatuses for printing the components, and, therefore, when the biological structure is prepared according to the instructions of the patient-adapted surgical plan, the bone-facing surfaces of the implant components will substantially match the prepared surfaces of the biological structure.

In addition to one or more of the surgical repair system components (e.g., implant, trial implant, instrument, surgical plan) expressly included in embodiments described above, various embodiments can also include providing one or more patient-adapted, physical anatomical models. Such patient-adapted anatomical models may be manufactured from, for example, any one or more of the various materials described above as suitable for use in manufacturing surgical repair system components, depending upon, for example, an intended use of a particular model. In some embodiments, for example, one or more anatomical models may be manufactured out of a metal (e.g., printed by a DMLS printing apparatus, cast, and/or machined) and/or in some embodiments, one or more anatomical models may be manufactured out of a polymer (e.g., printed by an SLS printing apparatus).

In various embodiments, one or more patient-adapted, physical anatomical models may be produced along with other patient-adapted components of a surgical repair system utilizing, at least in part, one or more forms of the same derived patient-adapted surface model. Patient-adapted, physical anatomical models can be selected and/or designed to provide a representation of various portions, aspects, states, and/or conditions of a patient's anatomy. For example, such models may include a representation of a surface or surfaces comprising one or more types of tissue (e.g., cartilage, bone, cortical bone, trabecular bone, subchondral bone). Likewise, patient-adapted models can provide a representation of an anatomical surface of a patient in a substantially healthy or at least partially diseased state, and likewise can provide corrected and/or uncorrected forms (e.g., as described elsewhere herein) of an anatomical surface. Furthermore, patient-adapted models can provide a representation of one or more anatomical surfaces prior to a surgical treatment and/or a representation of one or more planned resected-bone surfaces. Accordingly, the (or at least one of the) particular patient-adapted surface model(s) (e.g., model of planned resected-bone, model of a joint-facing surface), as well as the form or forms of the particular surface model(s) (e.g., positive, negative, corrected, uncorrected), utilized to produce an anatomical model can vary and may depend upon, for example, the intended purpose or function of the anatomical model and/or the intended surface(s) and form(s) thereof to be represented.

In some embodiments, one or more patient-adapted, physical anatomical models may be produced to be provided to a surgeon and/or medical facility that will perform the corresponding surgical procedure. For example, in some embodiments, one or more patient-adapted physical models (e.g., of one or more current surfaces of the joint, of one or more planned resected-bone surfaces) may be provided to a surgeon as part of providing specifications for a selected and/or designed surgical repair system in advance of the surgery, to facilitate the surgeon's review and/or provision of feedback associated with the specifications. Alternatively or in addition, one or more such patient-adapted, physical models may be included as part of the surgeon-approved and/or final patient-adapted surgical repair system or kit supplied for performing the surgery. In some embodiments, such models may be utilized by a surgeon as a reference or resource in planning for and/or in performance of the surgery. Furthermore, optionally, one or more of such patient-adapted, physical models may be provided to the patient.

Alternatively or in addition to producing one or more patient-adapted, physical anatomical models for provision to the surgeon, healthcare facility, and/or patient, in some embodiments, one or more patient-adapted, physical models may be utilized during one or more steps of manufacturing (e.g., finishing steps, inspection steps, post-3D printing steps, as discussed further below) one or more of the other patient-adapted components of the surgical repair system. For example, in some embodiments, a patient-adapted physical model may include one or more planned-resected bone surfaces, and accordingly, a patient-adapted implant (and/or trial implant) component may be placed on the model (e.g., such that bone-facing surfaces of the implant component are positioned to engage corresponding cut-bone surfaces of the model). Placing the implant component on the model may provide for verification and/or assessment of the fit of the bone-facing surfaces of the implant component with respect to the planned bone cuts. Further, with the implant component placed thereon, the model can provide a means for holding and/or securing the implant component (e.g., with respect to manufacturing equipment) for one or more manufacturing steps (e.g., finishing steps, inspection steps, post-3D printing steps). Similarly, in some embodiments, a patient-adapted physical model may include one or more surfaces corresponding to one or more current (and optionally, uncorrected) joint surfaces, and in some such embodiments, one or more patient-adapted instrument components may be placed on or against a patient-adapted surface of the model in order to evaluate the engagement and/or registration therewith of a corresponding patient-adapted surface of the instrument.

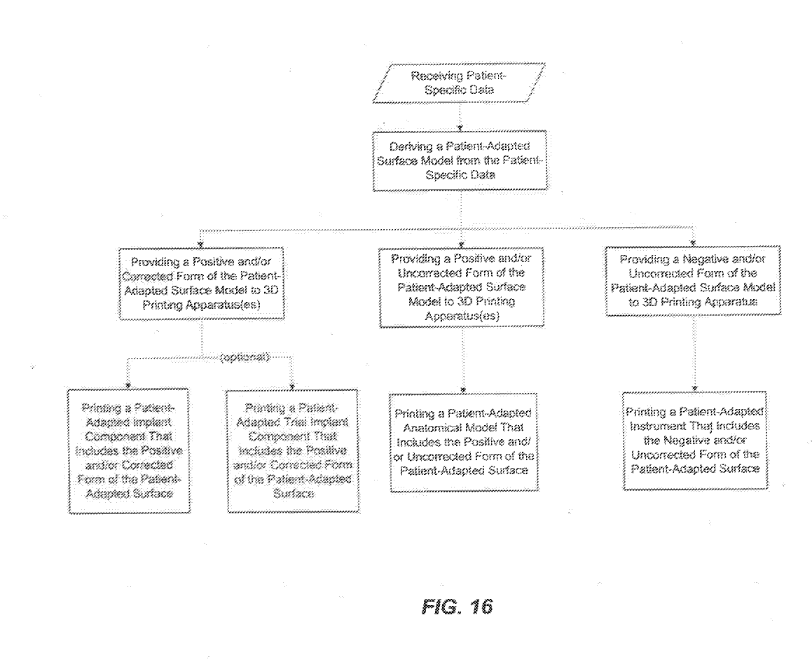

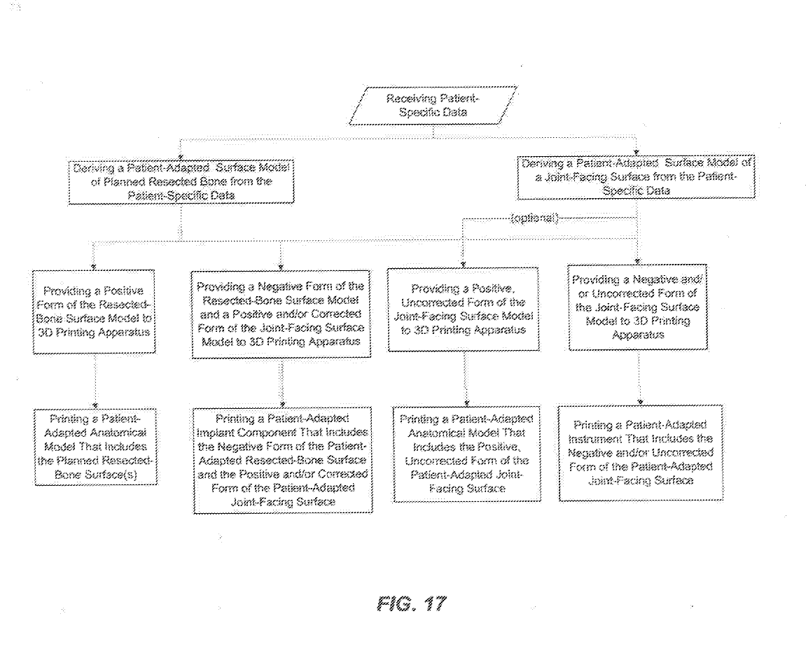

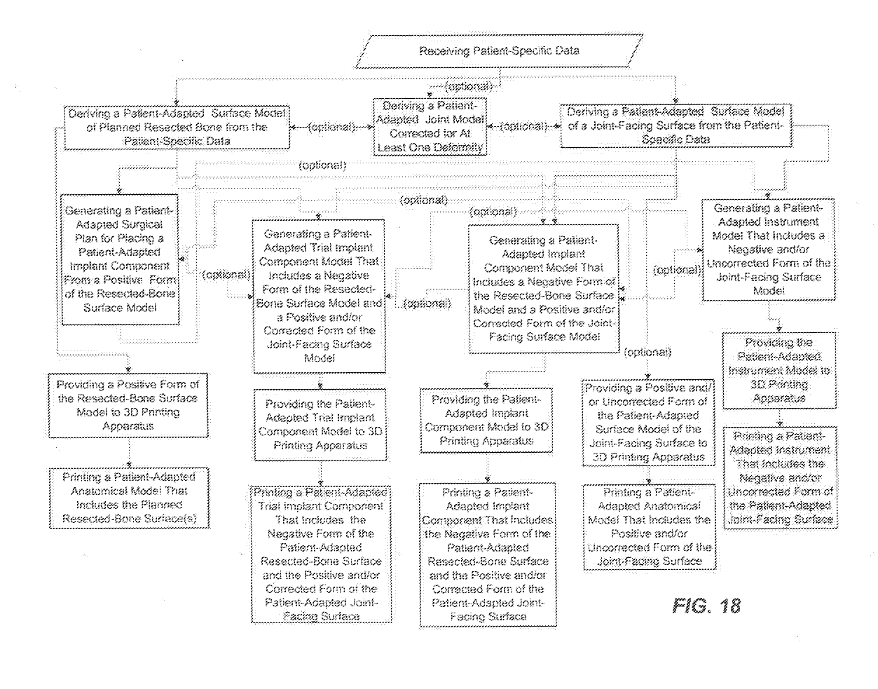

By way of example, in some embodiments, a patient-adapted, physical anatomical model, a patient-adapted implant component, a patient-adapted instrument, and, optionally, a patient-adapted trial implant component may each be produced utilizing, at least in part, one or more forms of the same derived patient-adapted surface model. As shown in FIG. 16, for example, some such embodiments can include receiving patient-specific data and deriving at least one patient-adapted surface model from, at least in part, a portion of the patient-specific data. In some embodiments, a positive and/or corrected form of the derived patient-adapted surface model may then be provided to one or more 3D printing apparatuses for printing the patient-adapted implant component, and, optionally, the patient-adapted trial implant component. Further, a negative and/or uncorrected form of the patient-adapted surface model may be provided to a 3D printing apparatus for printing the patient-adapted instrument. And, in some embodiments, a positive and/or uncorrected form of the patient-adapted surface model may be provided to a 3D printing apparatus for printing the patient-adapted anatomical model.

### Post-3D Printing Manufacturing Steps

While in some embodiments, one, some, or all components of a surgical repair system that are directly produced by 3D printing may not require additional manufacturing steps, in various embodiments, one or more additional processing and/or finishing steps may be needed for one or more components that have been 3D printed. Depending, for example, on the component and/or the 3D printing process used, one or more of a variety of additional processing and/or finishing steps may be needed and/or advantageous. In some embodiments, such steps can include, for example, one or more of coating, filling, heat treating, re-melting, hot isostatic pressing (“HIP”), annealing, machining, grinding, surface finishing, polishing, drag finishing, machining, bead blasting, grit blasting, and/or inspecting the components. As referred to herein, “post-3D printing” manufacturing or production steps can include both steps performed after all 3D printing of a component has been completed and/or steps performed after any of one or more 3D printing steps involved with production of the component has been completed (i.e., in some embodiments, post-3D printing production steps can include steps performed after an initial 3D printing step but prior to one or more subsequent 3D printing steps involved with production of the component).

Alternatively or in addition to utilizing one or more patient-adapted surface models in steps leading up to and/or including the physical 3D printing of one or more components of a surgical repair system, forms of the same patient-adapted surface model(s) may be utilized for one or more post-3D printing manufacturing steps. In some embodiments, utilizing forms of such available patient-adapted surface models for one or more subsequent manufacturing steps (e.g., additional processing and/or finishing steps) can have a variety of advantages, including, for example, enabling increased speed and/or accuracy of the performance of the steps. Accordingly, manufacturing time and/or costs may be reduced, in addition to the improvements achievable through various methods of 3D printing patient-adapted surgical repair system components, discussed above. Furthermore, quality of individual components of the surgical repair systems produced may be improved.

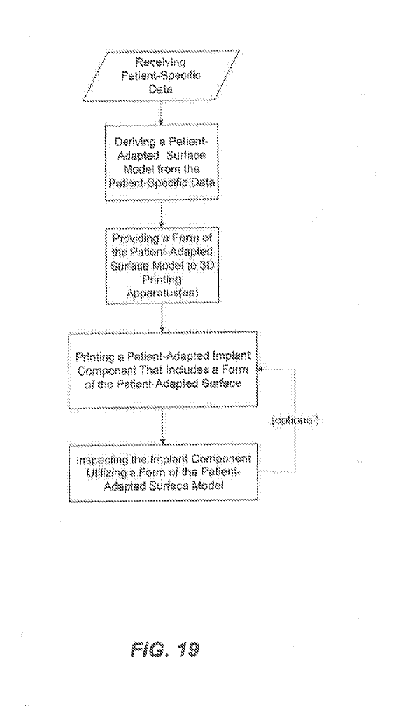

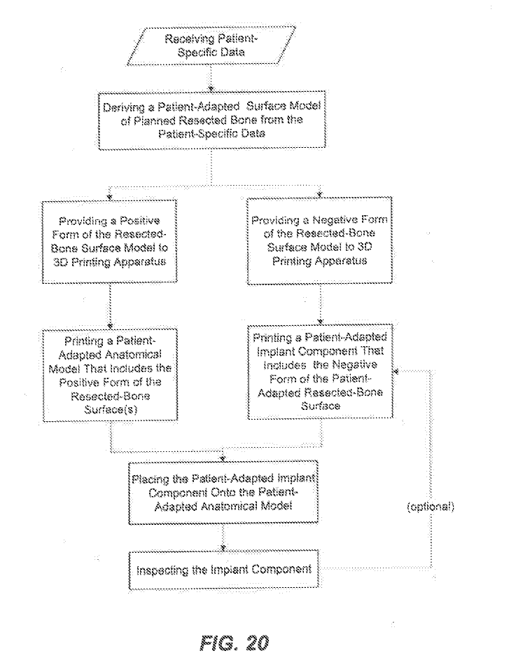

By way of example, in some embodiments, a form or forms of the same patient-adapted surface model may be used, at least in part, for both printing a component and inspecting the component produced. For example, an implant component may first be printed consistent with steps provided in embodiments described above. As shown in FIG. 19, this can include receiving patient-specific data associated with a joint or biological structure of a patient to be treated; deriving at least one patient-adapted surface model from, at least in part, a portion of the patient-specific data; providing a form of the at least one derived patient-adapted surface model to one or more 3D printing apparatuses; and printing a patient-adapted implant component that includes a form of the patient-adapted surface. Subsequently, the printed patient-adapted implant component may be inspected.

In some embodiments, at least a portion of the inspection process may include inspecting at least a portion of the surface of the implant component that corresponds to the derived patient-adapted surface, and thus, utilizing a form of the patient-adapted surface model may facilitate this process. For example, the corresponding surface, or one or more portions thereof, of the physical implant component may be analyzed and compared to the patient-adapted surface model to identify and/or determine the degree of any deviations of the physical surface from the modeled surface. Furthermore, optionally, in some embodiments, if any deviations identified by the inspection exceed predetermined allowable values, the patient-adapted implant component may be re-printed. In some such embodiments, one or more of the parameters of the 3D printing process may be adjusted and/or optimized for the re-printing in order to minimize and/or eliminate one or more deviations identified during the inspection process.

As will be appreciated, the same or similar steps described in the preceding paragraphs may be employed for printing and inspecting other components (e.g., trial implants, instruments) of a surgical repair system alternatively or in addition to implant components. Moreover, just as embodiments described above included utilizing forms of the same derived patient-adapted surface model to print multiple components of a surgical repair system, in some embodiments forms of the same derived patient-adapted surface model may be used to inspect one or more of the physical components produced.

Many additive manufacturing processes and 3D printing methods require and/or can be improved by the use of support structures during the printing, or part-build, process. Support structures can be necessary or helpful to support portions of a component for a variety of reasons. In some cases, the geometry of the part may not be able to stand on its own and/or portions of material may benefit from support during localized melting and/or curing that occurs as part of the printing process. In some cases, a part being built (or portions thereof) may need to be connected to a build platform by supports in order to absorb the weight of the part and/or the mechanical and thermal loads that occur during the build process. For some processes, supports may help absorb internal stresses that may occur during the cooling of the part. Similarly, for some processes, supports may help to dissipate heat after melting of a powder material. In addition, the use of support structures can anchor the manufactured part within the manufacturing equipment, preventing the part from uncontrolled movement and/or rotation/displacement during the manufacturing process, which could potentially ruin and/or degrade the quality of the part.

While support structures may be necessary during printing of components (e.g., implant components, trial implant components, instrument components, anatomical models), such supports generally must be removed to produce each component in its final form. In various embodiments described herein, removal of support structures can be performed at a variety of times after a component has been printed (e.g., immediately after printing of the components, prior to applying finishing processes to a component, after one or more finishing steps have been completed but prior to one or more additional finishing steps, or after all other finishing steps have been completed). Furthermore, removal of distinct support structures (and/or distinct portions of the same support structure) may be performed at separate times (e.g., one or more support structures may be removed prior to a particular finishing step and one or more additional support structures may be removed subsequent to the particular finishing step).

Removal of support structures from a printed component may be manual, automated, or semi-automated. For example, in some cases, manual techniques may be utilized for removing support structures. As part of the manufacturing process, an individual may remove support structures from a printed component by hand and/or utilizing one or more appropriate tools (e.g., snips, shears, saws, diagonal cutters, razors, knives, chisels, pry bars, torque levers, pliers, vices, grinders, sanders). Alternatively or in addition, the removal of at least some support structures may be semi-automated or automated (e.g., utilizing automated manufacturing equipment). For example, information regarding the position, shape, size, or attachment location of one or more support structures relative to a printed component (e.g., relative to one or more reference or registration surfaces or features of the printed component) can be provided to automated manufacturing equipment, such that when the component is positioned and/or fixed in a known position and/or orientation relative to the equipment, the automated manufacturing equipment can accurately engage, sever, and/or remove support structures from the component. Automated manufacturing equipment that may be used for removing support structures can include one or more industrial robots and/or one or more computer controlled manufacturing devices configured to apply, for example, a saw, laser, knife, high-pressure water jet, and/or twisting other mechanical force to a component, support structure, and/or attachment feature therebetween.

For standard (i.e., non-patient-adapted) components, which may comprise a single or limited number of configurations and/or sizes, the position, shape, size, and/or attachment location of one or more support structures printed with the components may be the same (or at least the same within each of the limited number of configurations and/or sizes). Thus, it may be possible and not cost-prohibitive to have tools, machines, instructions, and/or additional manufacturing components that are customized for use in removing one or more of such support structures from standard components because the same customized tools, machines, instructions, and/or additional manufacturing components can be used for all (or at least all of a particular configuration and/or size) of the standard components. In the case of at least some patient-adapted components, however, every component may have a different shape, size, etc., and accordingly, the number, position, shape, size, and/or attachment location of support structures associated with printing such components may vary and/or be entirely unique. Therefore, it may not be possible or practical to have tools, machines, instructions, and/or additional manufacturing components that are customized for removal of support structures from a given patient-adapted component, as it may be for standard components.

To account for this, one of several techniques provided herein may be utilized to facilitate and/or enable automated or semi-automated removal of support structures form patient-adapted components. In some embodiments, while at least important and/or critical aspects of the patient-adapted component may still be uniquely derived from patient-specific information, such components may be selected and/or designed with one or a limited number of dimensions and/or attachment points that are standardized. Inclusion of one more such standardized dimensions and/or attachment points for support structures may enable utilizing means for support structure removal that are the same or similar to those available for standard components (e.g., tools, machines, instructions, and/or additional manufacturing components that are customized based on the properties of the standard features to enable use for substantially all components having such standard features). Additionally or alternatively, in some embodiments, a mechanism for scanning (e.g., optically, mechanically) printed components to provide information to differentiate support structures from portions of the component itself may be utilized in conjunction with one or more automated manufacturing devices to provide information and/or guidance needed for the manufacturing device to remove support structures from various different patient-adapted components.

Alternatively or in addition, in some embodiments disclosed herein, automation and/or semi-automation of the removal of support structures from patient-adapted components can be facilitated and/or enabled by providing a form or forms of derived patient-adapted surface models associated with the particular component for utilization by automated manufacturing equipment. By way of example, a form or forms of the same patient-adapted surface model may be used, at least in part, for both printing a component and removing all or at least a portion of one or more support structures from the corresponding printed, physical component structure. Such automation or partial automation may help achieve improved speed of removal, improved accuracy of removal, and/or to obviate the need for one or more additional surface finishing steps after removal of the supports.

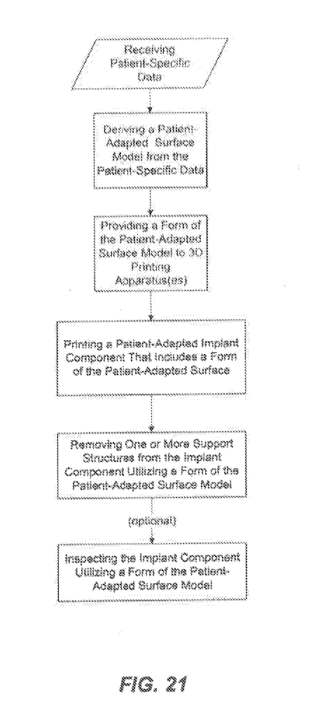

For example, an implant component may first be printed consistent with steps provided in embodiments described above. As shown in FIG. 21, this can include receiving patient-specific data associated with a joint or biological structure of a patient to be treated; deriving at least one patient-adapted surface model from, at least in part, a portion of the patient-specific data; providing a form of the at least one derived patient-adapted surface model to one or more 3D printing apparatuses; and printing a patient-adapted implant component that includes a form of the patient-adapted surface. Then supports (or portions thereof) may be removed by automated manufacturing equipment (e.g., an industrial robot and/or computer-controlled manufacturing devices) from the corresponding printed, physical implant component structure utilizing a form of the patient-adapted surface model. Optionally, after removing one or more support structures, some embodiments can include inspecting the implant component utilizing a form of the patient-adapted model.

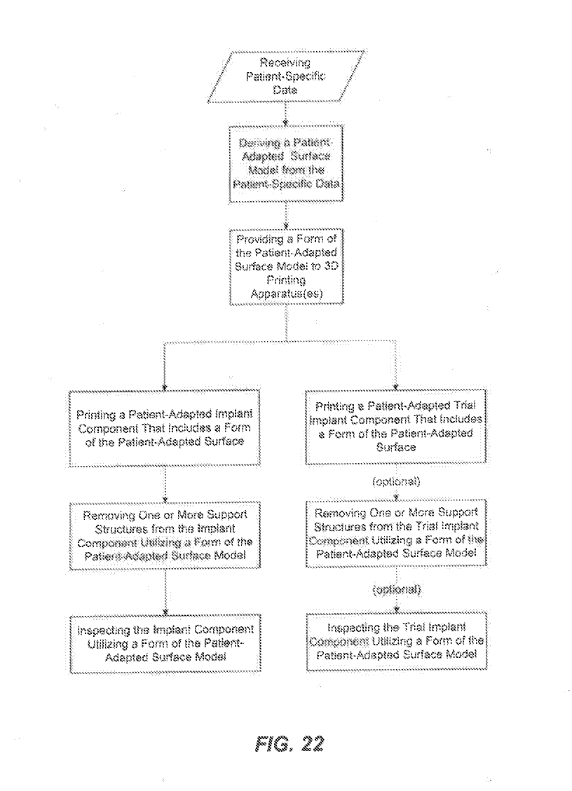

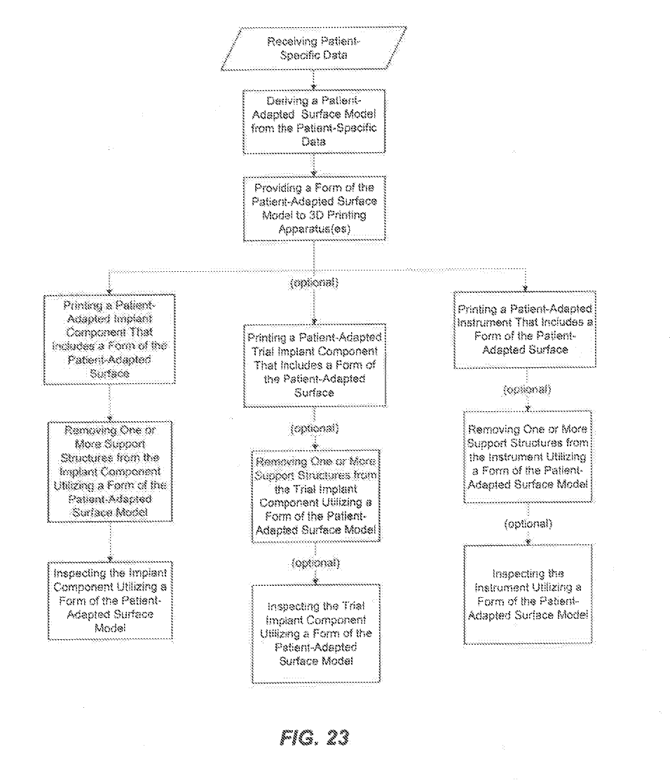

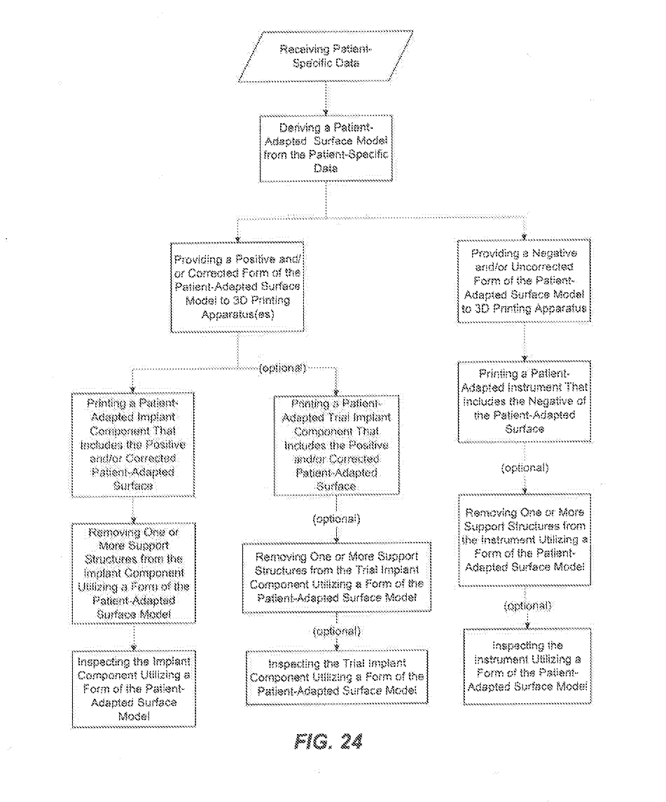

In various embodiments, the same or similar steps may be employed for manufacturing one or more other components (e.g., trial implants, instruments) of a surgical repair system. Moreover, similar to some embodiments described above that included utilizing forms of the same derived patient-adapted surface model to print multiple components of a surgical repair system, in some embodiments, forms of the same derived patient-adapted surface model may be utilized in printing of and/or removing support structures from multiple components of a surgical repair system. For example, as shown in FIG. 22, in some embodiments, a form or forms of a derived patient-adapted surface model used for printing of and removing support structures from an implant component may also be used for printing a patient-adapted trial implant component that includes a form of the patient-adapted surface and, optionally, removing one or more support structures from the trial implant component utilizing a form of the patient-adapted surface model. Likewise, as shown in FIG. 23, in some embodiments, a form or forms of a derived patient-adapted surface model used for printing of and removing support structures from an implant component may also be used for printing a patient-adapted instrument that includes a form of the patient-adapted surface. Some embodiments can include providing a positive and/or corrected form (e.g., as discussed elsewhere herein) of a derived patient-adapted surface model for printing a patient-adapted implant component, removing one or more support structures from the printed implant component utilizing a form of the patient-adapted surface model, and providing a negative and/or uncorrected form of the patient-adapted surface model for printing a patient-adapted instrument, as disclosed in FIG. 24. Furthermore, such embodiments, for example, as depicted in FIGS. 23 and 24, may optionally further include removing one or more support structures from the printed instrument utilizing a form of the patient-adapted surface model, printing a trial implant component that includes a form of the patient-adapted surface (e.g., the positive and/or corrected form), and/or removing one or more support structures from the trial implant component utilizing a form of the patient-adapted surface model.

A form or forms of various types of patient-adapted surface models may be utilized in a variety of manners to facilitate removal of support structures from a corresponding printed, patient-adapted component structure. “Utilizing” a form of a patient-adapted surface model in removing one or more support structures can generally include, for example, any one or more of: utilization of a form (e.g., CAD files) of the surface model directly; utilization of a model of the component that includes a form of the surface model; utilization of a form of the surface model along with information regarding support structures (e.g., relative location of attachment points) generated for the component printing, utilizing a model that include both the patient-adapted surface model and at least one surface model of a support structure generated; and/or deriving information and/or instructions for automated manufacturing equipment from, at least in part, one or more of the forgoing. In some embodiments, a patient-adapted surface model utilized may correspond to at least a portion of a surface from which a support structure must be removed. For example, an automated manufacturing device (e.g., CNC machine) may be instructed and/or controlled to cut (or otherwise detach) a support structure from the patient-adapted surface without substantially cutting (or otherwise altering) the remainder of the patient-adapted surface by using a form of the patient-adapted surface model and/or information derived, at least in part, therefrom to accurately determine the location, orientation, and/or path for the cut (and/or the position of other portions of the patient-adapted surface, alterations to which preferably and/or must be avoided).

Additionally or alternatively, in some embodiments, a patient-adapted surface model corresponding to a surface to which no support structures are attached may be utilized to facilitate removal of support structures. For example, at least a portion of a patient-adapted surface may be engaged, supported, or otherwise secured and/or registered by and/or to an automated manufacturing device. And thus, a form of the corresponding patient-adapted surface model and/or information derived, at least in part, therefrom can be utilized in one or more of engaging, positioning, and/or registering at least a portion of the printed, physical component structure in a known position and/or orientation by and/or with respect to the automated manufacturing device. This in turn can facilitate and/or enable removal of support structures, which are attached to one or more other surfaces of the component, by the automated manufacturing device.

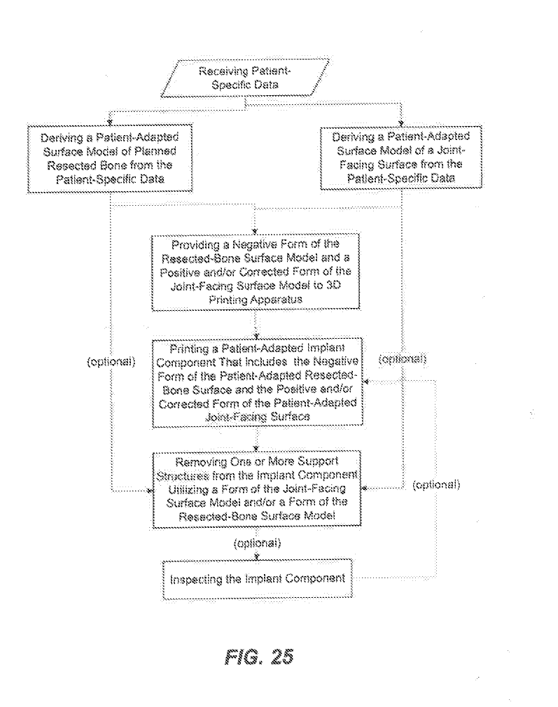

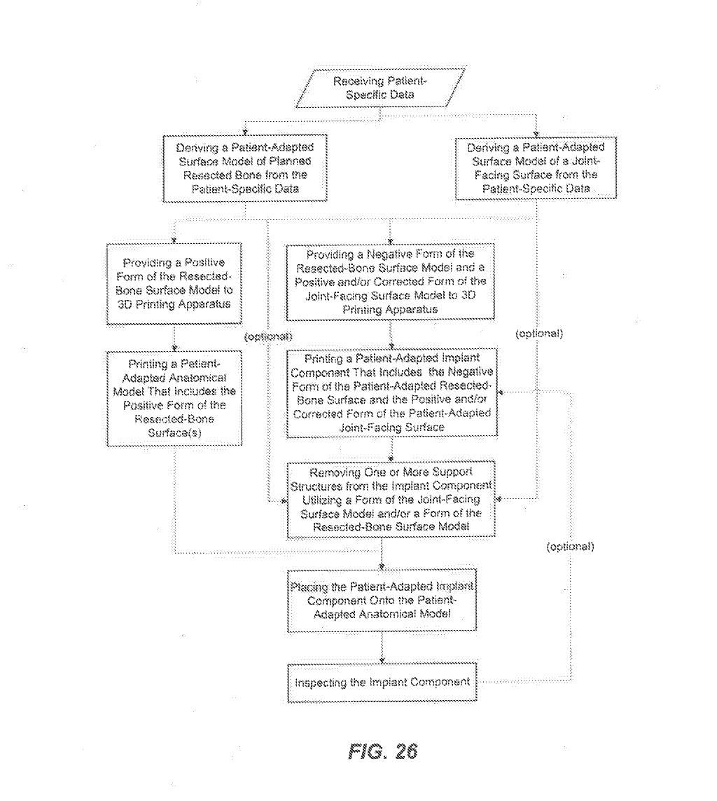

In embodiments that include printing implant and/or trial implant components, a form or forms of one or more derived patient-adapted surface models of a joint-facing surface and/or of planned resected-bone surface may be utilized in removing support structures from corresponding printed, patient-adapted component structures. For example, as illustrated in FIG. 25, some embodiments can include receiving patient-specific data associated with a joint of a patient to be treated; deriving a patient-adapted surface model of a joint-facing surface from, at least in part, a portion of the patient-specific data; and deriving a patient-adapted surface model of a planned resected-bone surface from, at least in part, a portion of the patient-specific data. A negative form of the resected-bone surface model and a positive and/or corrected form of the joint-facing surface model may be provided to a 3D printing apparatus, and an implant component including the patient-adapted joint-facing and resected-bone surfaces may be printed. As discussed above, as part of the printing process, one or more support structures may also be printed and attached to the printed implant component. In removing one or more of such support structures, a form of the patient-adapted joint-facing surface model, a form of the patient-adapted resected-bone surface model, or both may be utilized. As discussed above, in some embodiments, utilizing one or more of these patient-adapted surface models may facilitate utilizing automated and/or semi-automated removal of the support structures from such patient-adapted implant components.

In some embodiments, one or more support structures generated in printing an implant component may be attached to and/or otherwise contact a joint facing surface of the implant component. At least some portions of a joint facing surface may comprise surfaces intended for implant articulating. For example, outer, joint facing surfaces of a femoral implant component (particularly those substantially opposite to the inner, bone-facing surfaces) typically form articulating surfaces that interact with polymer and/or metal surfaces of opposing implant components. Accordingly, at least in some embodiments, the dimensionality and/or shape of such surfaces can be critical or important features of the implant. As such, at least in some cases, imprecision that may be associated with manual detachment and removal of support structures that extend from such surfaces may necessitate additional processing and/or finishing of the articulating surfaces, or in some cases, where such steps cannot sufficiently correct for defects imparted my the manual detachment process, re-printing of the implant component may be necessary. In at least some embodiments disclosed herein that enable automated or semi-automated removal of such support structures from patient-adapted implant components, the need for at least some such additional processing and/or finishing steps, as well as the potential need to re-print an implant component, may be substantially diminished or eliminated. For example, the precision of the detachment achievable by automated manufacturing equipment, optionally, in conjunction with information from one or more forms of patient-adapted surface models, may result in a detachment of the support structure that leaves substantially no remaining additional material on the intended articulating surface.

Alternatively or in addition, in various embodiments, one or more support structures generated in printing an implant component may be attached to and/or otherwise contact a bone-facing surface of the implant component. In some embodiments, at least a portion of a bone-facing surface of an implant component (e.g., at least a portion of the inner, bone-facing surfaces of a femoral implant component, including, for example, cement pockets) may comprise surfaces that do not require significant “finishing” after manufacture (and/or the need for such finishing may not be desired by the manufacturer). Thus, imprecision in detachment and removal of support structures attached to such surfaces may be undesirable to the extent, for example, that it necessitates additional processing and/or finishing of such surfaces. In some embodiments, such additional processing and/or finishing may be difficult to perform (e.g., the surfaces may recessed and/or obstructed by other surfaces and/or structures) and/or may simply introduce additional, unnecessary expense. Thus, various embodiments disclosed herein that enable automated or semi-automated removal of such support structures from patient-adapted implant components may be advantageous in reducing and/or eliminating the need for at least some such additional processing and/or finishing steps of at least some bone-facing surfaces.

Moreover, in addition or alternatively, in some embodiments, one or more support structures generated in printing an implant component may be attached to and/or otherwise contact one a peripheral or edge surface of an implant component. At least in some embodiments, a peripheral or edge surface of an implant may comprise a surface that connects or is otherwise disposed between an articulating surface and a bone-facing surface of an implant component and/or may comprise a surface located along a periphery of a bone-facing surface and disposed in a plane substantially perpendicular to a plane in which the bone-facing surface is disposed. In some cases, peripheral or edge surfaces of an implant component may be preferred surfaces for attachment of support structures. As a peripheral or edge surface may not be bone-facing, it may comprise a portion of a joint-facing surface of an implant (e.g., in addition to an articulating surface portion of the implant). Accordingly, in some embodiments, at least a portion of a form of a patient-adapted surface model of a joint-facing surface of an implant component may comprise information regarding the size, shape, and/or location of at least a portion of a peripheral or edge surface. Additionally or alternatively, a surface model and/or information regarding a peripheral or edge surface may be derived for adjacent bone-facing and articulating surface models. Thus, in some embodiments, a surface model of and/or information derived regarding a peripheral or edge surface may be utilized in automated and/or semi-automated removal of support structures attached to the peripheral or edge surface.

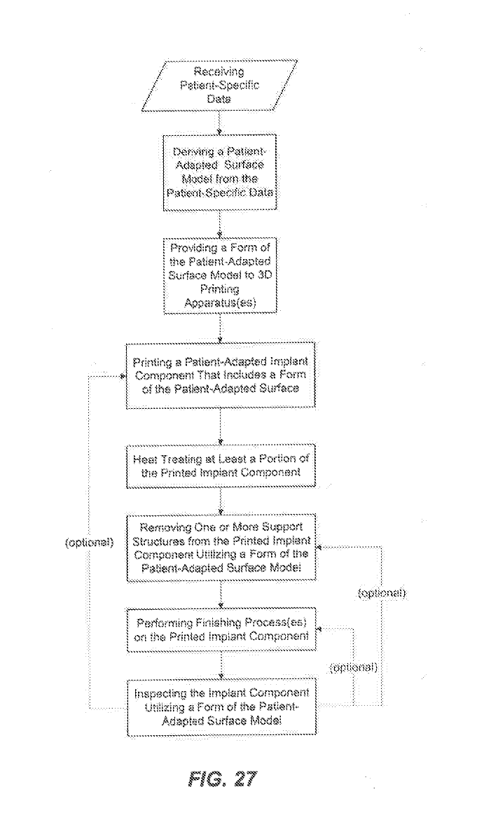

Furthermore, in various embodiments, including, for example, as depicted in FIGS. 21-25, one or more printed components may, optionally, be inspected utilizing a form or forms of one or more derived patient-adapted surface models (e.g., as described above), after one or more support structures have been removed from the printed implant component. In some embodiments, after inspection of the component, if any deviations identified exceed predetermined allowable values, one or more subsequent manufacturing steps may be initiated. For example, as depicted in FIG. 27, depending on the results of the inspection (e.g., the location and magnitude of deviations of surfaces of the component from surface models), optionally, further finishing processes may be performed on the printed implant component, further removal of one or more support structures from the printed component may be undertaken, and/or the component may be re-printed.

### Patient-Specific Data

Various embodiments of manufacturing surgical repair systems disclosed herein include acquiring and/or receiving patient-specific data. Patient-specific data can be obtained non-invasively and/or preoperatively, and/or patient-specific data can be obtained intraoperatively. In some embodiments, patient-specific data can include imaging data collected from the patient. Any current or future imaging modalities, including, for example, x-ray imaging, digital radiography, tomosynthesis, cone beam CT, non-spiral or spiral CT, non-isotropic or isotropic MRI, scintigraphy, SPECT, PET, ultrasound, laser imaging, photo-acoustic imaging, elastography (e.g., using MRI, ultrasound, or x-ray) may be used to acquire patient-specific data. Imaging data may be acquired in 2D or 3D (e.g., via 3D ultrasound or 3D MRI) and with or without the use of intra-articular or intravenous contrast agents.

Patient-specific data may additionally or alternatively include data from other sources and/or derived from imaging data. For example, in some embodiments, patient-specific data can include one dimensional, two-dimensional, and/or three-dimensional measurements obtained using mechanical means, laser devices, electromagnetic or optical tracking systems, molds, and/or materials applied to the articular surface that harden as a negative match of the surface contour. Measurements obtained can include, but are not limited to, one or more of length, width, height, depth and/or thickness; curvature (e.g., curvature in two dimensions, curvature in three dimensions, and/or a radius or radii of curvature); shape (e.g., two-dimensional shape, three-dimensional shape, contour); area (e.g., surface area and/or surface contour); perimeter shape; and/or volume. In certain embodiments, measurements of biological features can include any one or more of the illustrative measurements identified in Table 4 of US 2012-0209394. Patient-specific data may also include joint kinematic measurements (e.g., using gait analysis, dynamic and/or load-bearing imaging), bone density measurements, bone porosity measurements, identification of damaged or deformed tissues or structures, and/or patient information, such as, for example, patient age, weight, gender, ethnicity, activity level, and overall health status.

### Deriving Patient-Adapted Information

In some embodiments, received patient-specific data can be used, at least in part, to derive various types of patient-adapted (e.g., patient-specific and/or patient-engineered) information. For example, in some embodiments, measurements and/or surface models of relevant portions of a patient's anatomy can be derived from 2D and/or 3D patient-specific imaging data, as discussed above. Such derived measurements and/or models may include attributes, including, for example, length, width, height, depth and/or thickness; curvature (e.g., curvature in two dimensions, curvature in three dimensions, and/or a radius or radii of curvature); shape (e.g., two-dimensional shape, three-dimensional shape, contour); area (e.g., surface area and/or surface contour); perimeter shape; and/or volume of the relevant anatomy.

As discussed further below, derived patient-adapted information can be used in selecting and/or designing one or more components and/or component features of a surgical repair system. By way of example, some embodiments can include deriving one or more surface models of at least a portion of a patient's joint based, at least in part, on received patient-specific data. In some embodiments, the surface model(s) can be used in designing a new surgical repair system component and/or can be incorporated into an existing design of a component. For example, a patient-adapted surface model may be used in generating and/or modifying a model for a component. In some embodiments, a surface (or a portion thereof) of a model for a surgical repair system component may comprise the patient-adapted surface model. And thus, the surface (or a portion thereof) of the resulting manufactured component may substantially comprise the patient-adapted surface. Furthermore, patient-adapted surface models can be used in generating a surgical plan for placement of implant components.

As used herein, a “surface model” can comprise a representation of a portion of a surface or a representation of an entire surface (e.g., a portion of an articulating surface or an entire articulating surface of a biological structure). Likewise, a surface model can be used to refer to what may be considered a representation of a single surface (e.g., a single planar surface) or a representation of multiple surfaces (e.g., two or more planar surfaces). Furthermore, a surface model may be a representation of a closed surface or a representation of a non-closed surface. A surface model be a representation of a surface that defines the boundaries of a closed volume, or a surface model may not define a volume. A surface model may be one dimensional, two dimensional, or three dimensional. A surface model may be expressed, stored, and/or utilized in a variety of formats. For example, a surface model can be expressed as a mathematical expression, a topographical map, an image, a set of coordinate values, any other formats discussed herein, and/or any other current or future formats utilized by those of ordinary skill in the art. Similarly, a surface model can be in the format of an electronic or virtual model and/or a physical model. A surface model may be a representation of a surface comprising one or more types of material (e.g., metal, polymer) and/or tissue (e.g., cartilage, bone, cortical bone, trabecular bone, subchondral bone, cut bone).

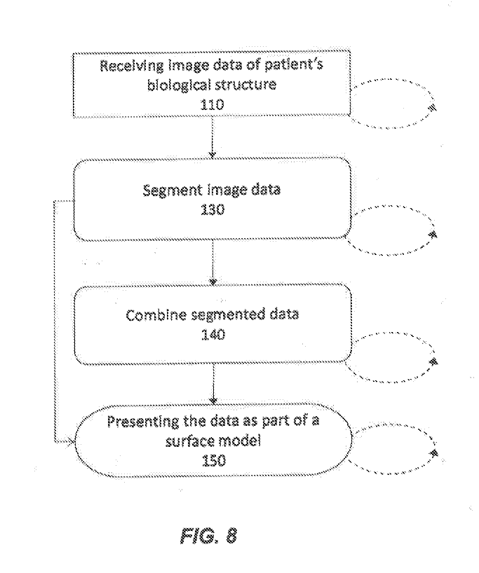

Various methods can be used to generate a surface model. As illustrated in FIG. 8, in certain embodiments, deriving a model of at least a portion of at least one surface of a patient's joint or other biological feature can include one or more of the steps of receiving image data of a patient's biological structure 110; segmenting the image data 130; combining the segmented data 140; and presenting the data as part of a model 150. Image data (2D and/or 3D) can be acquired from near or within the patient's biological structure of interest. For example, pixel or voxel data from one or more radiographic or tomographic images of a patient's joint can be obtained, for example, using computed or magnetic resonance tomography. Additionally or alternatively, other imaging modalities, including, for example, one or more of those identified above can be used. The acquired pixel or voxel data can then be received 110 for use in deriving a model. In this or a subsequent step, one or more of the pixels or voxels can be converted into one or a set of values. For example, a single pixel/voxel or a group of pixel/voxels can be converted to coordinate values, e.g., a point in a 2D or 3D coordinate system. The set of values also can include a value corresponding to the pixel/voxel intensity or relative grayscale color. Moreover, the set of values can include information about neighboring pixels or voxels, including, for example, information corresponding to relative intensity or grayscale color and/or information corresponding to relative position.

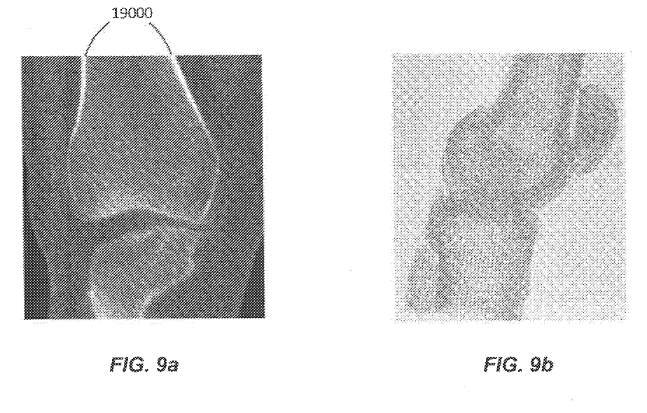

Then, the image data can be segmented 130 to identify those data corresponding to a particular biological feature of interest. For example, as shown in FIG. 9A, image data can be used to identify the edges of a biological structure, in this case, the surface outline for each of the patient's femur and tibia. As shown, the distinctive transition in color intensity or grayscale 19000 at the surface of the structure can be used to identify pixels, voxels, corresponding data points, a continuous line, and/or surface data representing the surface of the biological structure. This step can be performed automatically (for example, by a computer program operator function) or manually (for example, by a clinician or technician), or by a combination of the two.

Optionally, the segmented data can be combined 140. For example, in a single image, segmented and selected reference points (e.g., derived from pixels or voxels) and/or other data can be combined to create a line representing the surface outline of a biological structure. Moreover, as shown in FIG. 9B, segmented and selected data from multiple images can be combined to create a 3D representation of the biological structure. Alternatively, the images can be combined to form a 3D data set, from which the 3D representation of the biological structure can be derived directly using a 3D segmentation technique, for example an active surface or active shape model algorithm or other model based or surface fitting algorithm.

Then, the data can be presented as part of a surface model 150, such as, for example, a patient-adapted virtual surface model that includes the biological feature of interest. As will be appreciated by those of skill in the art, one or more of these steps 110, 130, 140, 150 can, optionally, be repeated as often as desired to achieve a desired result. Moreover, the steps can, optionally, be repeated reiteratively. Further, the process can, optionally, proceed directly from the step of segmenting image data 130 to presenting the data as part of a surface model 150. Alternatively, in certain embodiments, segmentation may not be necessary and data can be directly displayed and/or modeled using grayscale image information.

Optionally, 2D or 3D surface models (e.g., representations of a biological structure) can be refined, corrected, or otherwise manipulated. For example, a 3D representation may be smoothed, such as, for example, by employing a 3D polygon surface, a subdivision surface, a parametric surface, and/or a non-uniform rational B-spline (NURBS) surface. For a description of various parametric surface representations see, for example, Foley, J. D. et al., Computer Graphics: Principles and Practice in C; Addison-Wesley, 2nd edition (1995). Various methods are available for creating a parametric surface. For example, the 3D representation can be converted directly into a parametric surface, for example, by connecting data points to create a surface of polygons and applying rules for polygon curvatures, surface curvatures, and other features. Alternatively, a parametric surface can be best-fit to the 3D representation, for example, using publicly available software such as Geomagic® software (Research Triangle Park, N.C.). In various embodiments, a surface model for which at least a portion has been, or which is derived from a representation for which at least a portion has been, smoothed by one or more of the processes described herein can constitute a “corrected” surface model, as referred to elsewhere herein. Note, in some embodiments, a corrected surface model can comprise a surface model that has been refined, corrected, or altered in one or more ways (e.g., as discussed below) in addition to smoothing, or in some embodiments, no smoothing may be involved in deriving a corrected surface model.

In some embodiments, deriving a patient-adapted surface model may include selectively extracting one or more particular types of information from patient-specific imaging information. For example, some embodiments may optionally include extracting bone information, cartilage information, ligament information, meniscal information, labral information, and/or combinations thereof. Additionally or alternatively, some embodiments may optional involve extracting only non-diseased information from patient-specific imaging information, which in certain embodiments, could include excluding extraction of information regarding osteophytes. Furthermore, in some embodiments, diseased information may be optionally extracted.

In some instances derived patient-adapted information may substantially match the corresponding anatomical attribute of the patient (i.e., patient-specific derived information), while in other instances the derived information may be modified or corrected in one or more ways (e.g., adjusted to correct for deformities, as explained further below) relative to the corresponding anatomical attribute of the patient (i.e., patient-engineered derived information). The term “match,” as used herein, is envisioned to include one or both of a negative match, as in when a convex surface fits a concave surface, and a positive match, as in when one surface is identical to another surface. Various patient-adapted embodiments disclosed herein include utilizing derived patient-specific information, derived patient-engineered information, and/or combinations of both.

Selecting and/or Designing Models for Patient-Adapted Surgical Repair Systems

Component models (e.g., models for providing to 3D printing and/or other manufacturing apparatuses) of the surgical repair systems described herein can be selected and/or designed based, at least in part, received patient-specific data and/or derived patient-adapted information. For example, in some embodiments, one or more components of a surgical repair system can be selected from a library or database of models of systems of various sizes, including various medio-lateral (ML) antero-posterior (AP) and supero-inferior (SI) dimensions, curvatures and thicknesses, so that selected component models achieve desired parameters, as discussed further below. Alternatively or in addition, one or more features of an implant component model (and, optionally, a trial implant component, surgical plan, and/or guide tool) can be designed to include one or more patient-adapted features for a particular patient. In certain embodiments, one or more features of an implant component (and, optionally, a trial implant component, surgical plan, and/or guide tool) can be both selected and designed to include one or more patient-adapted features for the particular patient. For example, an implant component having features that achieve certain parameter thresholds but having other features that do not achieve other parameter thresholds (e.g., a blank feature, a smaller or larger feature) can be selected, for example, from a library of implant components. The selected component then can be further designed (e.g., virtually designed, manufactured, and/or subsequently machined) to alter the blank feature or smaller or larger feature to achieve the selected parameter (e.g., a patient-adapted dimension, a patient adapted surface).

The surgical repair systems described herein can be selected and/or designed to achieve various goals or parameters. For example, in some embodiments, a surgical repair system may be designed for an implant component to achieve a near anatomic fit or match with the surrounding or adjacent tissue (e.g., cartilage, subchondral bone, menisci). Additionally or alternatively, in some embodiments, a surgical repair system can be designed to reconstruct the shape of a healthy state of a biological structure (e.g., correct for cartilage disease or loss). Additional exemplary parameters for which models of surgical repair system components can be selected and/or designed to optimize are described in detail below. In various embodiments, using received patient-specific data and/or derived patient-adapted information, one or more aspects of an implant component, trial implant component, guide tool, and/or surgical plan (e.g., planned resection cuts) can be selected (e.g., from a virtual or physical library) and/or designed (e.g., virtually designed) to have one or more patient-adapted features, which facilitate the surgical repair system achieving the desired goals or parameters.

A variety of processes for selecting and/or designing components of a patient-adapted articular repair system can be used. For example, one or more selected implant component features and feature measurements; optionally, with one or more selected surgical plan features and feature measurements; and optionally, with one or more selected guide tool features and feature measurements can be generated and/or selected, altered, and/or assessed in series, in parallel, or in a combined process, to assess the fit between selected parameter goals or thresholds and the selected and/or designed features and feature measurements of the respective components. In some embodiments, the process can be iterative in nature. For example, one or more first parameters can be assessed and the related implant component and/or surgical plan features and feature measurements tentatively or conditionally can be determined. Next, one or more second parameters can be assessed and, optionally, one or more features and/or feature measurements determined. Then, the tentative or conditional features and/or feature measurements for the first assessed parameter(s) optionally can be altered based on the assessment and optional determinations for the second assessed parameters. The assessment process can be fully automated or it can be partially automated allowing for user interaction.

In some embodiments, during the selection and/or design process, different weighting can be applied to any of the parameters or parameter thresholds, including, for example, based on the patient's age, the surgeon's preference, or the patient's preference. Feedback mechanisms can be used to show a user or the software the effect that certain feature and/or feature measurement changes can have on desired changes to parameters values, e.g., relative to selected parameter thresholds. For example, a feedback mechanism can be used to determine the effect that changes in features intended to maximize bone preservation (e.g., implant component thickness(es), bone cut number, cut angles, cut orientations, and related resection cut number, angles, and orientations) have on other parameters such as limb alignment, deformity correction, and/or joint kinematic parameters, for example, relative to selected parameter thresholds. Accordingly, implant component features and/or feature measurements (and, optionally, surgical plan and guide tool features and/or feature measurements) can be modeled virtually and modified reiteratively to achieve an optimum solution for a particular patient.

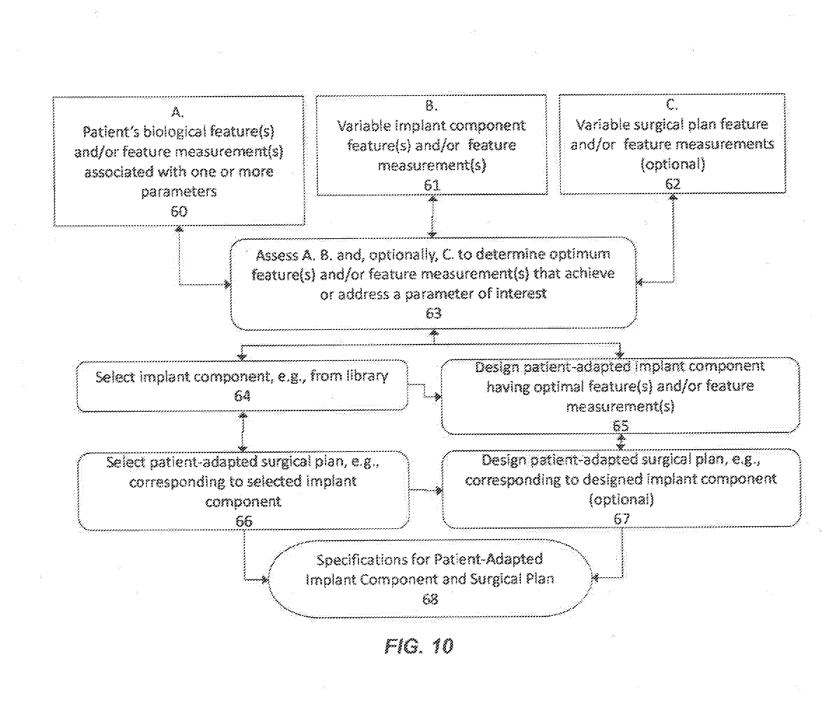

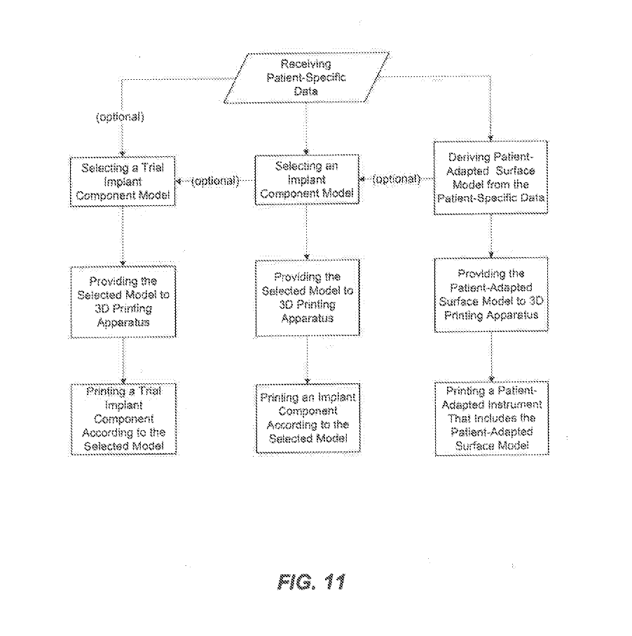

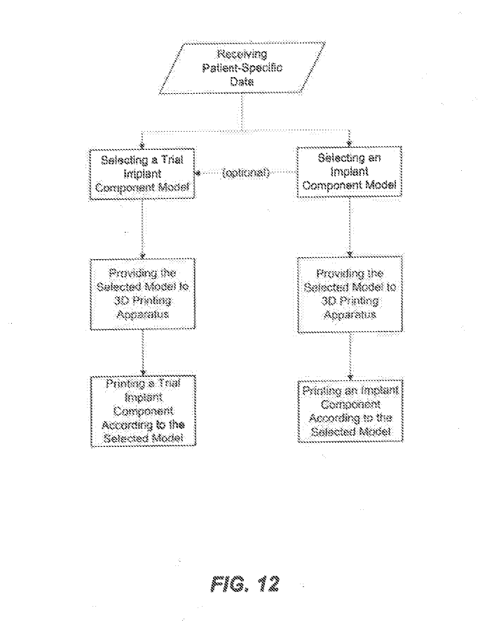

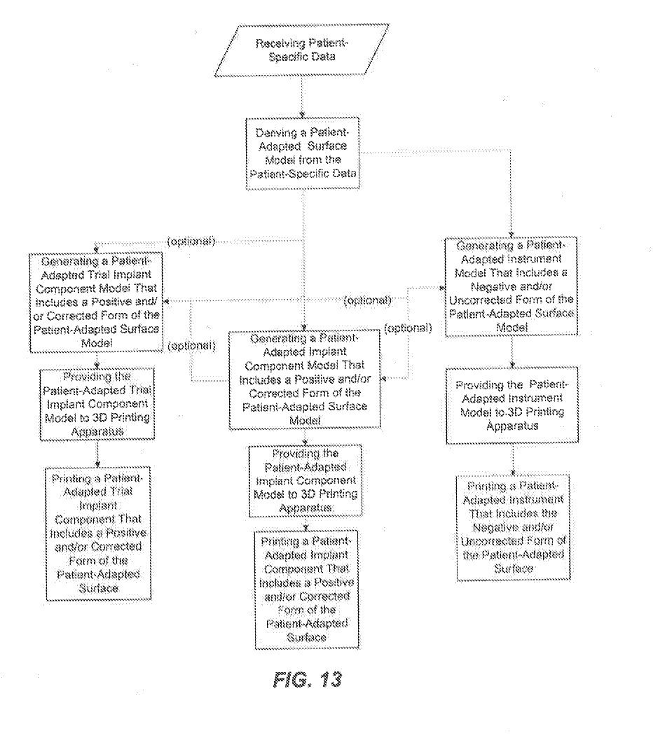

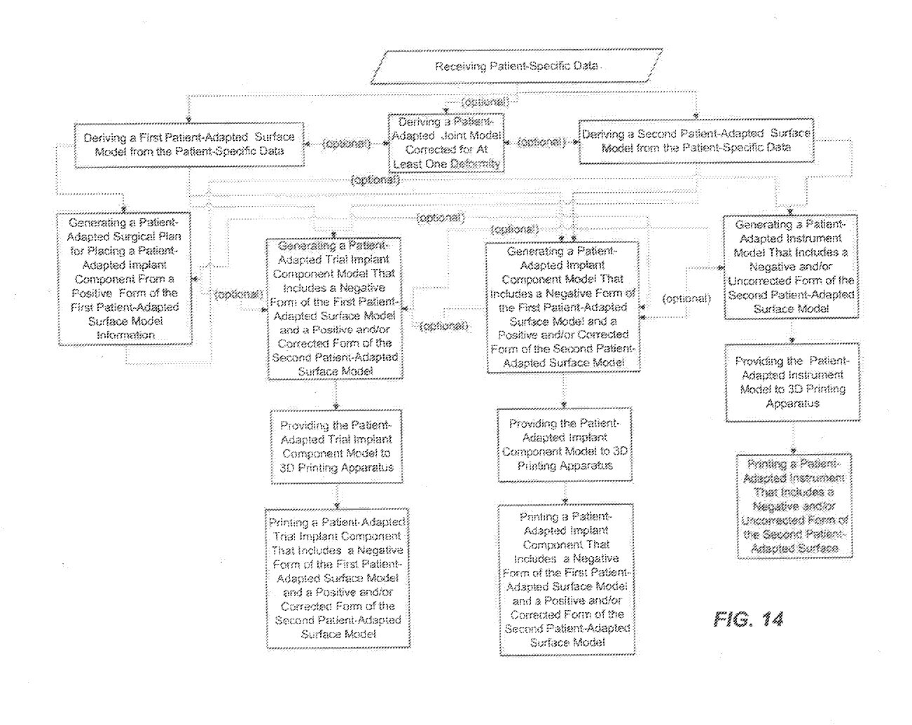

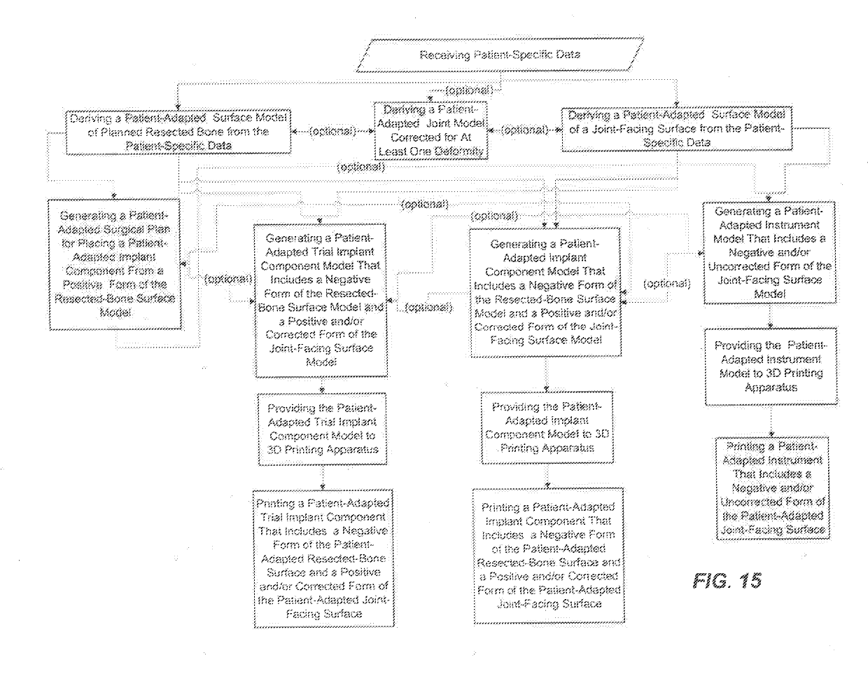

FIG. 10 is a flow chart illustrating one exemplary process of selecting and/or designing one or more implant component features and/or feature measurements, and, optionally, assessing and selecting and/or designing one or more surgical plan features and feature measurements, for a particular patient. Using the techniques described herein or those suitable and known in the art, one or more of the patient's biological features and/or feature measurements (e.g., patient-specific data, derived patient-adapted information, and/or derived patient-adapted surface models) are obtained 60. In addition, one or more variable implant component features and/or feature measurements are obtained 61. Optionally, one or more variable surgical plan features and/or feature measurements are obtained 62. Moreover, one or more variable guide tool features and/or feature measurements also can optionally be obtained. Each one of these step can be repeated multiple times, as desired.

The obtained patient's biological features and/or feature measurements, implant component features and/or feature measurements, and, optionally, surgical plan and/or guide tool features and/or feature measurements then can be assessed to determine the optimum implant component features and/or feature measurements, and optionally, surgical plan and/or guide tool features and/or feature measurements, that achieve one or more target or threshold values for parameters of interest 63 (e.g., by maintaining or restoring a patient's healthy joint feature). Once the one or more optimum implant component features and/or feature measurements are determined, the implant component(s) can be selected 64, designed 65, or selected and designed 64, 65. For example, a selected implant component having some optimum features and/or feature measurements can be further designed (e.g., using one or more CAD software programs or other specialized software to optimize additional features or feature measurements of the implant component). In some embodiments, this could include incorporating a derived patient-adapted surface model (e.g., of an articular surface of a joint) into, or in place of, a surface of a selected implant design.

Similarly, one or more surgical plan features and/or feature measurements can, optionally, be selected 66, designed 67, or selected and further designed 66, 67. For example, a surgical plan selected to have some optimum features and/or feature measurements can be designed further (e.g., using one or more CAD software programs or other specialized software to optimize additional features or measurements of the surgical plan). As an example, a surgical plan may be further designed such that resected bone surfaces substantially match optimized bone-facing surfaces of a selected and/or designed implant component. Moreover, optionally, one or more guide tool features and/or feature measurements can be selected, designed, or selected and further designed. For example, a guide tool having some optimum features and/or feature measurements can be designed further (e.g., using one or more CAD software programs or other specialized software) to optimize additional features or feature measurements of the guide tool. One or more manufacturing techniques described herein or known in the art can be used in the design step to produce the additional, optimized features and/or feature measurements, for example, to facilitate one or more resection cuts that, optionally, substantially match one or more optimized bone-facing surfaces of a selected and designed implant component. These processes can be repeated as desired.

As will be appreciated by those of skill in the art, the process of selecting and/or designing an implant component feature and/or feature measurement, resection cut feature and/or feature measurement, and/or guide tool feature and/or feature measurement can, optionally, be tested against patient-specific data obtained regarding the patient's biological features to ensure that the features and/or feature measurements are optimum with respect to the selected parameter targets or thresholds. Testing can be accomplished by, for example, superimposing the implant image over the image for the patient's joint. In a similar manner, load-bearing measurements and/or virtual simulations thereof may be utilized to optimize or otherwise alter a derived surgical repair system design.

Arriving at a combination of component features and/or feature measurements through the selection and/or design process that satisfy desired parameters produces specifications describing the selected and/or designed components (e.g., implant component, surgical plan, guide tools) 68. In some embodiments, these specifications may be in the form of one or more electronic models, or the specifications may be transferred into a software-directed computer system that performs a series of operations to transform and/or incorporate the data, and optionally other parameters, into one or more generated electronic models of the articular repair system components.

A variety of additional or alternative methods for selecting and/or designing one or more components of a surgical repair system may also be used. For example, in some embodiments, the physician, or other qualified individual can obtain a measurement of a biological feature (e.g., a target joint) and then directly select, design, or select and design a joint implant component having desired patient-adapted features and/or feature measurements.

In some embodiments, derived patient-adapted information, including measurements and/or models, can be used in modeling various aspects of a surgical repair system. For example, in certain embodiments, one or more patient-adapted surface models of a patient's joint can be used to generate a patient-engineered surgical plan, a patient-adapted guide tool design, a patient-adapted trial implant component, and/or a patient-adapted implant component design for a surgical procedure directly (i.e., without the one or more models themselves including one or more resection cuts, one or more drill holes, one or more guide tools, and/or one or more implant components). Additionally or alternatively, one or more models can be generated that includes at least one patient-adapted surface model of the patient's joint and includes or displays, as part of the model, one or more resection cuts, one or more drill holes (e.g., on a model of the patient's femur), one or more guide tools, one or more patient-adapted trial implant components, and/or one or more implant components. Moreover, in some embodiments, one or more resection cuts, one or more drill holes, one or more guide tools, one or more patient-adapted trial implant components, and/or one or more implant components can be modeled and selected and/or designed separate from a patient-adapted surface model of the patient's joint.

In some embodiments, at least some of the component specifications and/or electronic models may be transferred into a software-directed computer system that performs a series of operations to transform and/or incorporate the data into manufacturing specifications (e.g., for producing an implant component, a trial implant component, and/or a guide tool). In some embodiments, the electronic models and/or manufacturing specifications may then be transferred by a user and/or by electronic transfer into a software-directed computer system that directs one or more manufacturing instruments to produce one or more of the components from a starting material, such as a raw material or starting blank material. Optionally, in some embodiments, implant, implant trial, and/or guide tool design specifications and/or electronic models, as well as surgical plan specifications and/or associated electronic models may be transferred by a user and/or by electronic transfer into a software-directed computer system that performs a series of operations to transform the data into one or more surgical procedure specifications or instructions. In some instances the same or another software computer system may be configured to use the surgical procedure specifications or instructions to direct one or more automated surgical instruments, for example, a robot, to perform one or more surgical steps. In certain embodiments, one or more of these actions can be performed as steps in a single process by one or more software-directed computer systems.

### Trial Implant Components

As noted above, certain embodiments of surgical repair systems can include one or more trial implant components. A trial implant component can have one or more features that are substantially similar to and/or derived from a corresponding feature of a corresponding implant component. Such implant and trial implant component features can include, for example, dimensions (e.g., length, width, height, depth, thickness), curvature (e.g., curvature in two dimensions, curvature in three dimensions, and/or a radius or radii of curvature), shape (e.g., two-dimensional shape, three-dimensional shape, contour), area (e.g., surface area and/or surface contour), perimeter shape, volume, and/or fixation pins or pegs (e.g., pin/peg shape, length, width, location, orientation, number). In some embodiments, one or more features of the trial implant component that are substantially similar to the implant component can be features that are, at least in part, patient-adapted. Additionally or alternatively, in some embodiments, one or more of the features of the trial implant component that are substantially similar to the implant component can be features that are standard (i.e., non-patient-specific or “off-the-shelf” features).

Furthermore, in some embodiments, one or more trial implants may have one or more features that are substantially different from a corresponding feature of a corresponding implant component. In certain embodiments, the one or more features of the trial implant component that are substantially different from the implant component can be features that are, at least in part, patient-adapted. Additionally or alternatively, in some embodiments, one or more of the features of the trial implant component that are substantially different from the implant component can be features that are standard.

In some exemplary embodiments, a trial implant component can have an outer, joint-facing shape that is substantially the same as the outer, joint facing shape of the corresponding implant component. With such embodiments, a surgeon may use the trial implant component during the surgical procedure to evaluate function prior to final placement of the actual implant component. For example, with the trial implant component in its predetermined position and/or orientation within the joint, the surgeon may take the joint intraoperatively through a range of motion and thereby evaluate the joint function. In some embodiments, the surgeon may also assess, for example, ligament function, flexion balance, and/or extension balance with use of the trial implant component. In certain embodiments, the surgeon may also assess leg length or arm length with use of the trial implant component.

Additionally or alternatively, in some embodiments, a trial implant component can have an inner, bone-facing surface (or surfaces) that substantially differs (e.g., smaller, larger, location, orientation, clearance relative to dimensions of a biological structure), with respect to one or more features, relative to corresponding features of the corresponding implant component. For example, one or more features of an inner, bone-facing surface of a trial implant component may be substantially smaller than a corresponding feature of the implant component. In some embodiments, for example, a surgical plan may involve forming one or more pin holes or peg holes in a biological structure that are configured (e.g., sized, shaped, positioned, oriented) to receive corresponding pins and/or pegs on an inner, bone-facing surface of an implant component for fixation of the implant component. And a corresponding trial implant component can have one or more respectively corresponding pins and/or pegs that are smaller in one or more dimensions (e.g., diameter, length) than the corresponding dimension(s) of the pin or peg of the implant component. In this manner, ease of placement and/or removal of the trial implant component by the surgeon during the surgical procedure may be enhanced because the fit of the pins and/or pegs of the trial implant component within prepared holes in the biological structure will not be as tight, as compared to that of the final implant.

Similarly, in some additional or alternative embodiments, a surgical plan may involve forming one or more substantially planar bone cuts in a biological structure that are configured (e.g., sized, shaped, positioned, oriented) to match and support respectively corresponding inner, bone-facing surfaces of an implant component. And a corresponding trial implant component can also have corresponding substantially planar inner, bone-facing surfaces to match the bone cuts formed in the biological surface. But the planar, bone-facing surfaces of the trial implant can be substantially different in location compared to those of the actual implant component.

For example, one or more bone-facing surfaces of a trial implant can be located so as to be further away (relative to the corresponding bone-facing surfaces of the actual implant component) from the corresponding cut bone surface(s) when the trial implant component is in its predetermined position and/or orientation on the biological structure. Furthermore, in some embodiments, the distance between one or more bone-facing surfaces (e.g., distance between anterior and posterior bone-facing surfaces on a femoral trial implant component for a knee joint) can be greater (i.e., the surfaces are further apart) on the trial implant component than on the actual implant component. In some embodiments, this greater distance between bone-facing surfaces may again provide greater clearance and/or a less tight fit of the trial implant component on the prepared biological structure relative to the clearance and/or fit of the actual implant component, thereby enhancing the ease of positioning and/or removal of the trial implant component during the surgical procedure.

As another example, in some embodiments, a surgical plan may involve positioning at least a portion of an implant component in a recess, cavity, or defect (e.g., pre-existing or formed by a reamer and/or bur) in a biological structure. A corresponding portion of a trial implant component may have one or more corresponding dimensions that are smaller than that of the actual implant component. For example, a portion of an implant component to be positioned in a cavity may have one or more dimensions that are slightly larger than or substantially the same size as corresponding dimensions of the cavity in order to facilitate fixation of the portion of the implant within the cavity. But a corresponding trial implant may have dimensions that are slightly smaller than that of the cavity in order to enhance ease of positioning and/or removal of the trial implant component during the surgical procedure.

As discussed above, certain embodiments can include one or more guide tools having at least one patient-adapted bone-facing surface portion that substantially negatively-matches at least a portion of a biological surface at the patient's joint. The guide tool further can include at least one aperture for directing movement of a surgical instrument, for example, a securing pin or a cutting tool. One or more of the apertures can be designed to guide the surgical instrument to deliver a patient-optimized placement for, for example, a securing pin or resection cut. In addition or alternatively, one or more of the apertures can be designed to guide the surgical instrument to deliver a standard placement for, for example, a securing pin or resection cut. Alternatively, certain guide tools can be used for purposes other than guiding a drill or cutting tool. For example, balancing and trial guide tools can be used to assess alignment and/or fit of one or more implant components or inserts. As used herein, “guide tool,” “jig,” “instrument,” “tool,” and “surgical instrument” all generally refer to tools configured for use in a surgical procedure and thus, may be used interchangeably.

Certain embodiments can include a guide tool that includes at least one patient-adapted bone-facing surface that substantially negatively-matches, or references, at least a portion of a biological surface at the patient's joint. The patient's biological surface can include cartilage, bone, tendon, and/or other biological surface. In certain embodiments, patient-specific data such as imaging data of a patient's joint can be used to identify an area on the biological surface that is free or substantially free of cartilage to which a bone-facing surface may be designed to substantially negatively match. The area can be free of articular cartilage because it was never covered by cartilage or because the overlying cartilage has been worn away. For example, imaging data can be specifically used to identify areas of full or near full thickness cartilage loss. Alternatively, the area can be free of articular cartilage because an osteophyte has formed and is extending outside the cartilage. By selecting such a substantially cartilage-free surface area, the guide tool then can rest directly on the bone, e.g., subchondral bone, marrow bone, endosteal bone, and/or an osteophyte.

In certain embodiments, patient-specific data such as imaging test data of a patient's joint can be used to identify a contact area on an articular surface for deriving a surface model for at least a portion of a bone-facing surface of a guide tool to substantially negatively-match the contact area on a subchondral bone, endosteal bone, and/or bone marrow surface. While the area may be covered by articular cartilage, the guide tool surface area can be specifically designed to match the subchondral bone, endosteal bone, and/or bone marrow surface contact area. The guide tool can have one or multiple areas that substantially negatively-match one or multiple contact areas on the subchondral bone, endosteal bone, and/or bone marrow surface bone surface. Intraoperatively, the surgeon can elect to place the guide tool on the residual cartilage. Optionally, the surgeon then can mark the approximate contact area on the cartilage and remove the overlying cartilage in the marked area before replacing the guide tool directly onto the subchondral bone, endosteal bone, and/or bone marrow surface bone. In this manner, the surgeon can achieve more accurate placement of the guide tools that substantially negatively-matches subchondral bone, endosteal bone, and/or bone marrow surface bone.

In certain embodiment, an articular surface or the margins of the articular surface can include one or more osteophytes. A guide tool can be designed to rest on the articular surface, e.g., on at least one of normal cartilage, diseased cartilage, and subchondral bone, and it can include the surface shape of the osteophyte. In certain embodiments, patient-specific data such as imaging test data of a patient's joint can be used to derive a surface shape on the bone-facing surface of the guide tool that substantially negatively-matches the patient's articular surface including the osteophyte. In this manner, the osteophyte can provide additional anatomic referencing for placing the guide tool.

In certain embodiments, the osteophyte can be virtually removed from the joint on the 2D or 3D images and the contact surface of the guide tool can be derived based on the corrected surface model without the osteophyte. In this setting, the surgeon can remove the osteophyte intraoperatively prior to placing the guide tool.

If a subchondral bone surface is used to assess the patient's biological surface, a standard cartilage thickness (e.g., 2 mm), or an approximate cartilage thickness derived from patient-specific data (e.g., age, joint-size, contralateral joint measurements, etc.) can be used as part of the design for the guide tool, for example, to design the size and bone-facing surface of the guide tool. The standard or approximate cartilage thickness can vary in thickness across the assessed surface area.

In certain embodiments, a guide tool can include at least one feature for directing a surgical instrument to deliver a patient-engineered feature to the patient's biological structure, including, for example, a resection hole or a resection cut for engaging a patient-engineered implant peg or a patient-engineered implant bone-facing surface. Additionally or alternatively, in some embodiments, a guide tool can include at least one standard feature for directing a surgical instrument to deliver a standard feature to the patient's biological structure. For example, a guide tool may guide formation of a standard resection hole or standard resection cut for engaging a standard implant peg or a standard implant bone-facing surface.

### Parameters for Optimizing Models

As noted above, models for various components of the patient-adapted surgical repair systems disclosed herein can be selected and/or designed to optimize one or more parameters including, for example, one or more of (1) joint deformity correction; (2) limb alignment correction; (3) bone, cartilage, and/or ligaments preservation at the joint; (4) preservation, restoration, or enhancement of one or more features of the patient's biology, for example, trochlea and trochlear shape; (5) preservation, restoration, or enhancement of joint kinematics, including, for example, ligament function and implant impingement; (6) preservation, restoration, or enhancement of the patient's joint-line location and/or joint gap width; and (7) preservation, restoration, or enhancement of other target features. Such corrected models can then be utilized in producing the components of the repair system. Various features of a patient-adapted component can be designed or engineered based, at least in part, on patient-specific data and/or patient-adapted information to help meet any number of user-defined thresholds for the above-noted parameters. For example, features of an implant component that can be designed and/or engineered can include, but are not limited to, implant shape (external and internal), implant size, implant thickness, and/or any of the features specified in U.S. 2012-0209394 (e.g., listed in Table 1).

There are several advantages that a patient-adapted implant designed and/or engineered to meet or improve one of more of these parameters can have over a traditional implant. These advantages can include, for example: improved mechanical stability of the extremity; opportunity for a pre-primary or additional revision implant; improved fit with existing or modified biological features; improved motion and kinematics, and other advantages.

### Modeling and Addressing Joint Defects

In certain embodiments, the patient-specific data and/or patient-adapted information (e.g., surface models) described above can be processed (e.g., using mathematical functions) to derive virtual, corrected features and/or corrected surface models. Such corrected features and/or surface models may represent, for example, a restored, ideal, or desired feature. For example, one or more features, such as surface models or dimensions of a biological structure can be modeled, altered, added to, changed, deformed, eliminated, corrected and/or otherwise manipulated (collectively referred to herein as “variation” of an existing surface or structure within the joint). While described with respect to the knee, the embodiments described below can be applied to any joint or joint surface (e.g., a knee, hip, ankle, foot, toe, shoulder, elbow, wrist, hand, a spine or spinal joints) in the body.

Variation of the joint or portions of the joint can include, without limitation, variation of one or more external surfaces, internal surfaces, joint-facing surfaces, uncut surfaces, cut surfaces, altered surfaces, and/or partial surfaces, as well as variation of osteophytes, subchondral cysts, geodes or areas of eburnation, joint flattening, contour irregularity, and loss of normal shape. The surface or structure can be or reflect any surface or structure in the joint, including, without limitation, bone surfaces, ridges, plateaus, cartilage surfaces, ligament surfaces, or other surfaces or structures. The surface or structure derived can be an approximation of a healthy joint surface or structure or can be another variation. The surface or structure can be made to include pathological alterations of the joint. The surface or structure also can be made whereby the pathological joint changes are virtually removed in whole or in part.

For example, in some embodiments, the variation can be used to select and/or design a patient-adapted implant component and/or a patient-adapted trial implant component having an ideal or optimized feature or shape, in place of a deformed joint feature or shape. For example, in some instances, a corrected surface model of a portion of an implant and/or implant trial may approximate the shape of a corresponding portion of the patient's joint before he or she developed arthritis.

Alternatively or in addition, the variation can be used to select and/or design a patient-adapted surgical procedure to address the deformity or abnormality. For example, the variation can include surgical alterations to the joint, such as virtual resection cuts, virtual drill holes, virtual removal of osteophytes, and/or virtual building of structural support in the joint deemed necessary or beneficial to a desired final outcome for a patient and thereby produce a patient-adapted corrected surface model of the joint, based on the variations.

Corrections can be used to address osteophytes, subchondral voids, and other patient-specific defects or abnormalities. In the case of osteophytes, a corrected surface model for the bone or joint-facing surface of an implant component, trial implant component, and/or guide tool can be selected and/or designed with the osteophyte virtually removed. Alternatively, the osteophyte can be integrated into the shape of the bone or joint-facing surface of the implant component, trial implant component, and/or guide tool. In the case of building additional or improved structure, additional features of the implant component and/or trial implant component can be derived after corrected bone-facing surface models are generated. Optionally, a surgical plan and/or one or more guide tools can be selected and/or designed to reflect the correction and correspond to the implant component and/or trial implant component. Virtually any undesirable anatomical features or deformity, including (but not limited to) altered bone axes, flattening, potholes, cysts, scar tissue, osteophytes, tumors and/or bone spurs may be similarly virtually removed prior to planning implant design and placement.

Similarly, to address a subchondral void, a surface model for the bone-facing surface of an implant component can be derived after the void has been virtually removed (e.g., filled). Alternatively, the subchondral void can be integrated into the shape of the bone-facing surface of the implant component and/or the trial implant component. Optionally, a surgical strategy and/or one or more guide tools can be selected and/or designed to reflect the correction and correspond to the implant component and/or trial implant component.

In addition to osteophytes and subchondral voids, the methods, surgical strategies, guide tools, and implant components described herein can be used to address various other patient-specific joint defects or phenomena. In certain embodiments, correction can include the virtual removal of tissue, for example, to address an articular defect, to remove subchondral cysts, and/or to remove diseased or damaged tissue (e.g., cartilage, bone, or other types of tissue), such as osteochondritic tissue, necrotic tissue, and/or torn tissue. In such embodiments, the correction can include the virtual removal of the tissue (e.g., the tissue corresponding to the defect, cyst, disease, or damage) and the bone-facing surface of the implant component can be derived after the tissue has been virtually removed. In certain embodiments, the implant component can be selected and/or designed to include a thickness or other features that substantially matches the removed tissue and/or optimizes one or more parameters of the joint. Optionally, a surgical strategy and/or one or more guide tools can be selected and/or designed to reflect the correction and correspond to the implant component and/or trial implant component.

In certain embodiments, a correction can include the virtual addition of tissue or material, for example, to address an articular defect, loss of ligament stability, and/or a bone stock deficiency, such as a flattened articular surface that should be round. In certain embodiments, the additional material may be virtually added (and optionally then added in surgery) using filler materials such as bone cement, bone graft material, and/or other bone fillers. Alternatively or in addition, the additional material may be virtually added as part of the implant component, for example, by using a bone-facing surface and/or component thickness that match the correction or by otherwise integrating the correction into the shape of the implant component. Then, the joint-facing and/or other features of the implant can be derived. This correction can be designed to re-establish a near normal shape for the patient. Alternatively, the correction can be designed to establish a standardized shape or surface for the patient.

In certain embodiments, the patient's abnormal or flattened articular surface can be integrated into the shape of the implant component, for example, the bone-facing surface of the implant component can be designed to substantially negatively-match the abnormal or flattened surface, at least in part, and the thickness of the implant can be designed to establish the patient's healthy or an optimum position of the patient's structure in the joint. Moreover, the joint-facing surface of the implant component also can be designed to re-establish a near normal anatomic shape reflecting, for example, at least in part, the shape of normal cartilage or subchondral bone. Alternatively, it can be designed to establish a standardized shape.

In certain embodiments, models can be generated to show defects of interest in a patient's joint. For example, one model or set of models of a patient's joint can be generated showing defects of interest and, optionally, another model or set of models can be generated showing no defects (e.g., uncorrected and corrected models, respectively). Alternatively, or in addition, the same or additional models can be generated with and/or without resection cuts, guide tools, and/or implant components positioned in the model. Moreover, the same or additional models can be generated to show defects of interest that interfere with one or more resection cuts, guide tools, and/or implant components. Such models, showing defects of interest, resection cuts, guide tools, implant components, and/or interfering defects of interest, can be particularly useful as a reference or guide to a surgeon or clinician prior to and/or during surgery, such as, for example, in identifying proper placement of a guide tool or implant component at one or more steps in a surgery, and/or in identifying features of a patient's anatomy that he or she may want to alter during one or more steps in a surgery. Accordingly, such models that provide, for example, patient-adapted renderings of implant assemblies and defects of interest (e.g., osteophyte structures) together with bone models, can be useful in aiding surgeons and clinicians in surgery planning and/or during surgery.

The models can include virtual corrections reflecting a surgical plan, such as one or more of removed osteophytes, cut planes, drill holes, realignments of mechanical or anatomical axes. The models can include comparison views demonstrating the anatomical situation before and after applying the planned correction. The individual steps of the surgical plan can also be illustrated in a series of step-by-step images wherein each image shows a different step of the surgical procedure.

The models can be presented to the surgeon as a printed or digital set of images. In another embodiment, the models are transmitted to the surgeon as a digital file, which the surgeon can display on a local computer. The digital file can contain image renderings of the models. Alternatively, the models can be displayed in an animation or video. The models can also be presented as a 3D model that is interactively rendered on the surgeon's computer. The models can, for example, be rotated to be viewed from different angles. Different components of the models, such as bone surfaces, defects, resection cuts, axes, guide tools or implants, can be turned on and off collectively or individually to illustrate or simulate the individual patient's surgical plan. The 3D model can be transmitted to the surgeon in a variety of formats, for example in Adobe 3D PDF or as a SolidWorks eDrawing.

### Modeling Proper Limb Alignment

Proper joint and limb function can depend on correct limb alignment. For example, in repairing a knee joint with one or more knee implant components, optimal functioning of the new knee can depend on the correct alignment of the anatomical and/or mechanical axes of the lower extremity. Accordingly, an important consideration in designing and/or replacing a natural joint with one or more implant components is proper limb alignment or, when the malfunctioning joint contributes to a misalignment, proper realignment of the limb.

Certain embodiments described herein include utilizing patient-specific data to virtually determine in one or more planes one or more of an anatomic axis and a mechanical axis and the related misalignment of a patient's limb. The misalignment of a limb joint relative to the axis can identify the degree of deformity, for example, varus or valgus deformity in the coronal plane or genu antecurvatum or recurvatum deformity in the sagittal plane. Then, one or more aspects of a patient-adapted surgical repair can be designed to help correct the misalignment.

A patient's axis and misalignment can be derived from patient-specific data, such as, for example, imaging information acquired via one or more of the various imaging modalities and techniques described above. For example, data from the imaging information can be used to determine anatomic reference points or limb alignment, including alignment angles within the same and between different joints or to simulate normal limb alignment. Any anatomic features related to the misalignment can be selected and imaged. For example, in certain embodiments, such as for a knee or hip implant, the image acquisition can include data from at least one of, or several of, a hip joint, knee joint and ankle joint. The imaging information can be acquired from the patient in lying, prone, supine or standing position. The imaging acquisition can include only the target joint, or both the target joint and also selected data through one or more adjoining joints.

Using the image information, one or more mechanical or anatomical axes, angles, planes or combinations thereof can be determined. In certain embodiments, such axes, angles, and/or planes can include, or be derived from, one or more of a Whiteside's line, Blumensaat's line, transepicondylar line, femoral shaft axis, femoral neck axis, acetabular angle, lines tangent to the superior and inferior acetabular margin, lines tangent to the anterior or posterior acetabular margin, femoral shaft axis, tibial shaft axis, transmalleolar axis, posterior condylar line, tangent(s) to the trochlea of the knee joint, tangents to the medial or lateral patellar facet, lines tangent or perpendicular to the medial and lateral posterior condyles, lines tangent or perpendicular to a central weight-bearing zone of the medial and lateral femoral condyles, lines transecting the medial and lateral posterior condyles, for example through their respective centerpoints, lines tangent or perpendicular to the tibial tuberosity, lines vertical or at an angle to any of the aforementioned lines, and/or lines tangent to or intersecting the cortical bone of any bone adjacent to or enclosed in a joint. Moreover, estimating a mechanical axis, an angle, or plane also can be performed using image data obtained through two or more joints, such as the knee and ankle joint, for example, by using the femoral shaft axis and a centerpoint or other point in the ankle, such as a point between the malleoli.

As one example, if surgery of the knee or hip is contemplated, the imaging test can include acquiring data through at least one of, or several of, a hip joint, knee joint or ankle joint. As another example, if surgery of the knee joint is contemplated, a mechanical axis can be determined. For example, the centerpoint of the hip knee and ankle can be determined. By connecting the centerpoint of the hip with that of the ankle, a mechanical axis can be determined in the coronal plane. The position of the knee relative to said mechanical axis can be a reflection of the degree of varus or valgus deformity. The same determinations can be made in the sagittal plane, for example to determine the degree of genu antecurvatum or recurvatum. Similarly, any of these determinations can be made in any other desired planes, in two or three dimensions.

Exemplary methods for virtually aligning a patient's lower extremity are described below in Example 9 of U.S. 2012-0209394. In particular, Example 9 illustrates methods for determining a patient's tibial mechanical axis, femoral mechanical axis, and the sagittal and coronal planes for each axis. However, any current and future method for determining limb alignment and simulating normal knee alignment can be used.

Once the proper alignment of the patient's extremity has been determined virtually, one or more surgical steps (e.g., resection cuts) may be planned and/or accomplished, which may include the use of surgical tools (e.g., tools to guide the resection cuts), and/or implant components (e.g., components having variable thicknesses to address misalignment).

Modeling Cartilage Defects and/or Loss

In some embodiments, a near normal cartilage surface at the position of a cartilage defect can be reconstructed by interpolating a healthy cartilage surface across the cartilage defect or area of diseased cartilage, thereby deriving a corrected surface model. This can, for example, be achieved by describing the healthy cartilage by means of a parametric surface (e.g. a B-spline surface), for which the control points are placed such that the parametric surface follows the contour of the healthy cartilage and bridges the cartilage defect or area of diseased cartilage. The continuity properties of the parametric surface can provide a smooth integration of the part that bridges the cartilage defect or area of diseased cartilage with the contour of the surrounding healthy cartilage. The part of the parametric surface over the area of the cartilage defect or area of diseased cartilage can be used to determine the shape or part of the shape of the surgical repair system to match with the surrounding cartilage.

In another embodiment, a near normal cartilage surface (i.e., corrected surface model) at the position of the cartilage defect or area of diseased cartilage can be reconstructed using morphological image processing. For example, in a first step, the cartilage can be extracted from the electronic image using manual, semi-automated and/or automated segmentation techniques (e.g., manual tracing, region growing, live wire, model-based segmentation), resulting in a binary image. Defects in the cartilage appear as indentations that can be filled with a morphological closing operation performed in 2-D or 3-D with an appropriately selected structuring element. The closing operation is typically defined as a dilation followed by an erosion. For example, a dilation operator can set the current pixel in the output image to 1 if at least one pixel of the structuring element lies inside a region in the source image. An erosion operator can set the current pixel in the output image to 1 if the whole structuring element lies inside a region in the source image. The filling of the cartilage defect or area of diseased cartilage creates a new surface over the area of the cartilage defect or area of diseased cartilage that can be used to determine the shape or part of the shape of the surgical repair system to match with the surrounding cartilage or subchondral bone.

Cartilage loss in one compartment can lead to progressive joint deformity. For example, cartilage loss in a medial compartment of the knee can lead to varus deformity. In certain embodiments, cartilage loss can be estimated in the affected compartments. The estimation of cartilage loss can be done using an ultrasound MRI or CT scan or other imaging modality, optionally with intravenous or intra-articular contrast. The estimation of cartilage loss can be as simple as measuring or estimating the amount of joint space loss seen on x-rays. For the latter, typically standing x-rays are preferred. If cartilage loss is measured from x-rays using joint space loss, cartilage loss on one or two opposing articular surfaces can be estimated by, for example, dividing the measured or estimated joint space loss by two to reflect the cartilage loss on one articular surface. Other ratios or calculations are applicable depending on the joint or the location within the joint. Subsequently, a normal cartilage thickness can be virtually established on one or more articular surfaces by simulating normal cartilage thickness. In this manner, a normal or near normal cartilage surface can be derived. Normal cartilage thickness can be virtually simulated using a computer, for example, based on computer models, for example using the thickness of adjacent normal cartilage, cartilage in a contralateral joint, or other anatomic information including subchondral bone shape or other articular geometries. Cartilage models and estimates of cartilage thickness can also be derived from anatomic reference databases that can be matched, for example, to a patient's weight, sex, height, race, gender, or articular geometry(ies).

In certain embodiments, a patient's limb alignment can be virtually corrected by realigning the knee after establishing a normal cartilage thickness or shape in the affected compartment by moving the joint bodies, for example, femur and tibia, so that the opposing cartilage surfaces including any augmented or derived or virtual cartilage surface touch each other, typically in the preferred contact areas. These contact areas can be simulated for various degrees of flexion or extension.

### Maximizing Preservation of Tissue

In certain embodiments, resection cuts are optimized to preserve the maximum amount of bone for each individual patient, based on a series of two-dimensional images or a three-dimensional representation of the patient's articular anatomy and geometry and the desired limb alignment and/or desired deformity correction. Resection cuts on two opposing articular surfaces can be optimized to achieve the minimum amount of bone resected from one or both articular surfaces.

The resection cuts also can be designed to meet or exceed a certain minimum material thickness, for example, the minimum amount of thickness required to ensure biomechanical stability and durability of the implant. In certain embodiments, the limiting minimum implant thickness can be defined at the intersection of two adjoining bone cuts on the inner, bone-facing surface of an implant component.

Implant design and modeling also can be used to achieve ligament sparing, for example, with regard to the PCL and/or the ACL. An imaging test can be utilized to identify, for example, the origin and/or the insertion of the PCL and the ACL on the femur and tibia. The origin and the insertion can be identified by visualizing, for example, the ligaments directly, as is possible with MRI or spiral CT arthrography, or by visualizing bony landmarks known to be the origin or insertion of the ligament such as the medial and lateral tibial spines.

An implant system can then be selected or designed based on the image data so that, for example, the femoral component preserves the ACL and/or PCL origin, and the tibial component preserves the ACL and/or PCL attachment. The implant can be selected or designed so that bone cuts adjacent to the ACL or PCL attachment or origin do not weaken the bone to induce a potential fracture.

### Establishing Normal or Near-Normal Joint Kinematics

In certain embodiments, bone cuts and implant shape including at least one of a bone-facing or a joint-facing surface model of the implant can be designed or selected to achieve normal joint kinematics.

In certain embodiments, a computer program simulating biomotion of one or more joints, such as, for example, a knee joint, or a knee and ankle joint, or a hip, knee and/or ankle joint can be utilized. In certain embodiments, patient-specific imaging data can be fed into this computer program. For example, a series of two-dimensional images of a patient's knee joint or a three-dimensional representation of a patient's knee joint can be entered into the program. Additionally, two-dimensional images or a three-dimensional representation of the patient's ankle joint and/or hip joint may be added.

Alternatively, patient-specific kinematic data, for example obtained in a gait lab, can be fed into the computer program. Alternatively, patient-specific navigation data, for example generated using a surgical navigation system, image guided or non-image guided can be fed into the computer program. This kinematic or navigation data can, for example, be generated by applying optical or RF markers to the limb and by registering the markers and then measuring limb movements, for example, flexion, extension, abduction, adduction, rotation, and other limb movements.

### Joint Line Restoration

In certain embodiments, an implant component can be designed based on patient-specific data to include a thickness profile between its joint-facing surface and its bone-facing surface to restore and/or optimize the particular patient's joint-line location.

### Automation

Any one or more steps of the assessment, selection, and/or design of an articular repair system may be partially or fully automated, for example, using a computer-run software program and/or one or more robots. For example, processing of the patient data, the assessment of biological features and/or feature measurements, the assessment of implant component features and/or feature measurements, the optional assessment of resection cut and/or guide tool features and/or feature measurements, the selection and/or design of one or more features of a patient-adapted implant component, and/or the implantation procedure(s) may be partially or wholly automated. For example, patient data, with optional user-defined parameters, may be inputted or transferred by a user and/or by electronic transfer into a software-directed computer system that can identify variable implant component features and/or feature measurements and perform operations to generate one or more virtual models and/or implant design specifications, for example, in accordance with one or more target or threshold parameters. Implant selection and/or design data, with optional user-defined parameters, may be inputted or transferred by a user and/or by electronic transfer into a software-directed computer system that performs a series of operations to transform the data and optional parameters into one or more implant manufacturing specifications.

What is claimed is:

1. A method of making a surgical repair system for a patient, the method comprising:
receiving patient-specific data regarding the patient;
deriving a patient-adapted surface model of planned resected bone from the patient-specific data;
deriving a patient-adapted surface model of a joint-facing surface from the patient-specific data;
providing a negative form of the resected-bone surface model and a positive and/or corrected form of the joint-facing surface model to a 3D printing apparatus;
printing a patient-adapted implant component that substantially includes the negative form of the patient-adapted resected-bone surface and the positive and/or corrected form of the patient-adapted joint-facing surface; and
removing one or more support structures from the implant component utilizing a form of the joint-facing surface model and/or a form of the resected-bone surface model.

2. The method of claim 1, wherein the removing one or more support structures from the implant component comprises utilizing the negative form of the resected-bone surface model.

3. The method of claim 1, wherein the removing one or more support structures from the implant component comprises utilizing the positive and/or corrected form of the patient-adapted joint-facing surface.

4. The method of claim 1, further comprising inspecting the implant component utilizing a form of the joint-facing surface model and/or a form of the resected-bone surface model;

5. The method of claim 1, further comprising providing a positive form of the resected-bone surface model to a 3D printing apparatus; and printing a patient-adapted anatomical model that includes the positive form of the resected-bone surface.

6. The method of claim 5, further comprising placing the patient-adapted implant component onto the patient-adapted anatomical model; and inspecting the implant component.

7. A method of making a surgical repair system for a patient, the method comprising:
receiving patient-specific data regarding the patient;
deriving a patient-adapted surface model from the patient-specific data;
providing a form of the patient-adapted surface model to at least one 3D printing apparatus;
printing a patient-adapted implant component that substantially includes the form of the patient-adapted surface model; and
removing one or more support structures from the implant component utilizing, at least in part, the first form of the patient-adapted surface model.

8. The method of claim 7, further comprising inspecting the implant component utilizing, at least in part, the first form of the patient-adapted surface model.

9. The method of claim 7, wherein the form of the patient-adapted surface model comprises a positive and/or corrected form of the patient-adapted surface model.

10. The method of claim 7, wherein the form of the patient-adapted surface model comprises a negative and/or uncorrected form of the patient-adapted surface model.

11. A method of making a surgical repair system for a patient, the method comprising:
receiving patient-specific data regarding the patient;
deriving a patient-adapted surface model from the patient-specific data;
providing a positive and/or corrected form of the patient-adapted surface model to at least one 3D printing apparatus;
providing a negative and/or uncorrected form of the patient-adapted surface model to at least one 3D printing apparatus;
printing a patient-adapted implant component that substantially includes the positive and/or corrected form of the patient-adapted surface;
printing a patient-adapted instrument that substantially includes the negative and/or uncorrected form of the patient-adapted surface; and
printing a patient-adapted trial implant component that substantially includes the positive and/or corrected form of the patient-adapted surface.
